(19) United States
(12) Patent Application Publication
(10) Pub. No.: US 2010/0234246 A1
(43) Pub. Date: Sep. 16, 2010

(54) METHOD FOR PRODUCING PEPTIDE LIBRARIES AND USE THEREOF

(75) Inventors: Eva Jung, Frankfurt am Main (DE); Manfred Hendlich, Frankfurt am Main (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/529,780

(22) PCT Filed: Mar. 4, 2008
(86) PCT No.: PCT/EP08/01687

§ 371 Date: Mar. 9, 2010

(30) Foreign Application Priority Data
Mar. 13, 2007 (DE) 10 2007 011 912.9

(51) Int. Cl. C40B 40/10; C07K 7/06; C07K 7/08; C07K 14/00; G06F 15/18; G06N 5/02

(52) U.S. Cl. 506/18; 530/329; 530/328; 530/327; 530/326; 530/325; 530/324; 706/12; 706/54

(57) ABSTRACT

Screening libraries of peptides in different assays offers an opportunity to simultaneously interrogate intracellular signaling pathways, create reagents to further the understanding of the pathway, and to create novel forms of therapies.

Many, if not all, biologically active peptides (e.g. peptide hormones) have profound effects both in health and disease, either by growth stimulating roles, growth inhibitory roles, or the regulation of critical metabolic pathways.

The present invention is directed to novel bioactive peptides, an in silico method to identify these peptides and a peptide library containing these peptides.

### FIELD OF THE INVENTION

The present invention relates to the field of computational biochemistry and computer aided design of bioactive peptides. It combines methods used in biological sequence analysis, bioinformatics data mining, information representation and classification algorithms using supervised learning. In addition it relates to the design of peptide libraries and the use of bioactive peptides for biomedical research.

### BACKGROUND OF THE INVENTION

A primary goal of drug discovery today is to identify biologically active molecules that have practical clinical utility. Many, if not all, biologically active peptides (e.g. peptide hormones) have profound effects both in health and disease, either by growth stimulating roles, growth inhibitory roles, or the regulation of critical metabolic pathways.

Peptide hormones are produced as precursors in different cell types and organs like glands, neurons, intestine, brain, etc. Peptide hormones are initially synthesized as larger precursors, or prohormones, and may acquire a number of post-translational modifications during transportation through the ER and Golgi stacks. They are processed and transported to their final destination to act as active substances (first messengers) to trigger a cellular response by binding to a cell surface receptor.

Peptide hormones are the key messengers in many physiological processes including regulation of production; growth; water and salt metabolism; temperature control; cardiovascular, gastrointestinal, and respiratory control; behavior; memory; and affective states.

Peptide hormones play a key role in physiological processes that are relevant to many areas of biomedical research such as diabetes (Insulin), blood pressure regulation (Angiotensin), anemia (Erythropoietin-α), multiple sclerosis (Interferon-β), obesity (Leptin) and others.

Therefore, novel bioactive peptides have the potential to be used as therapeutic polypeptides, targets for drug intervention, ligands to discover relevant targets (eg. GPCR deorphaning) or biomarkers to monitor diseases.

Peptide libraries have successfully been used to identify bioactive peptides, including antimicrobial peptides, receptor agonists and antagonists, ligands for cell surface receptors, protein kinase inhibitors and substrates, T-cell epitopes, peptides binding to MHC molecules and peptide mimotopes of receptor binding sites. Peptide libraries can be categorized according to their origin in gene- and synthetic based libraries (Falciani et al., 2005).

In gene based libraries the combinatorial positions within the polypeptides are introduced at the DNA level that encodes the sequence of the target polypeptide in order to introduce diversity. In contrast to the gene based libraries, synthetic libraries achieve their diversity at the level of chemical synthesis.

Many peptide libraries are based on one scaffold or use a random combinatorial approach to generate different polypeptide primary structures.

The disadvantage of both approaches is that the combination of the 20 naturally occurring amino acids allows the construction of polypeptides which are most variable and account for a very large number of different structures. To give an example on how many different structures can be obtained, consider the 160,000 different primary structure possibilities for a peptide containing only 4 amino acids.

There was a need to provide an accurate and high-throughput method to significantly reduce the potential number of structures in a peptide library, to enable the processing of large amounts of data and to distinguish between peptides that have an activity in vivo and peptides that do not have an activity in vivo.

The object of the present invention solves the problem of the prior art. The present invention relates to a method to construct novel bioactive peptide hormone libraries using a bioinformatics strategy. A support vector machine (SVM) algorithm is used to identify bioactive peptides. This method allows to discovering potential bioactive peptide hormones in silico searching the human proteome by taking advantage of the conserved protein features and short motifs present in peptide hormone precursors. While these features are common to peptide hormones and are responsible for their maturation, there is, surprisingly, very little sequence similarity between peptide hormone precursors that would allow a database search on the protein sequence level alone (e.g. BLAST, FASTA). However, combinations of co-occurring protein features and motifs for post-translational modifications in peptide hormone precursors (e.g. short protein sequence length of precursor, signal peptide, disulfide bonds, amidation sites, sulfation sites, glycosylation sites, etc) can be used to discover novel peptide hormones with a high specificity.

### SUMMARY OF THE INVENTION

One subject-matter of the present invention refers to a method for identifying bioactive peptides using a binary support vector machine (SVM) based algorithm in a computer-based system, wherein:
a) a SVM algorithm is trained to learn to distinguish between bioactive and non bioactive peptides, said training comprising the steps of:
   a₁) generating vectors with 49 dimensions, each dimension resulting from the calculation of a molecular descriptor value, for a set of labelled known bioactive and labelled known non bioactive peptides, where the labels indicate whether the peptide is, respectively, bioactive or non bioactive;
   a₂) transferring the vector data generated in step a₁) to the SVM based algorithm, said algorithm calculating the optimal hyperplane that separates the vectors corresponding to the bioactive peptides and the non bioactive peptides, respectively;
b) protein sequences are provided from a publicly available human protein database;
c) secondary structure and cleavage sites within a protein sequence provided in step b) are predicted using computational methods; a set of 7 molecular descriptors is calculated based on said prediction step resulting in the generation of peptide fragments;
d) a set of 42 molecular descriptors corresponding to the physico-chemical properties of the peptide fragments generated in step c) is calculated;
e) the calculated values from step c) are transformed into scaled values between 0 and 1 to generate dimensions 1 to 7 of a 49-dimension-vector for each peptide fragment and the calculated values from step d) are transformed into scaled values between 0 and 1 to generate dimensions 8 to 49 of said vector for each peptide fragment;
f) the vectors generated in the step e) are presented to the trained SVM algorithm from step a) to measure the distance of each vector to the hyperplane calculated in step a₂); and
g) each peptide fragment is classified as bioactive peptide or non bioactive peptide, according to the distance measured in step f).
In general, dimensions 1 to 7 generated in step e) are the following: Dimension 1: N-terminal ProP score; Dimension 2: N-terminal Hmcut score; Dimension 3: N-terminal fragment; Dimension 4: C-terminal ProP score; Dimension 5: C-terminal Hmcut score; Dimension 6: C-terminal Hamid score; Dimension 7: C-terminal fragment; and dimensions 8 to 49 generated in step e) are the following: Dimension 8: Percentage of acidic amino acids (E, N, Q) per polypeptide; Dimension 9: Percentage of positively charged amino acids (R, H) per polypeptide; Dimension 10: Percentage of aromatic amino acids (F, Y, W) per polypeptide; Dimension 11: Percentage of aliphatic amino acids (G, V, A, I) per polypeptide; Dimension 12: Percentage of Proline per polypeptide; Dimension 13: Percentage of reactive amino acids (S, T) per polypeptide; Dimension 14: Percentage of Alanine per polypeptide; Dimension 15: Percentage of Cysteine per polypeptide; Dimension 16: Percentage of Glutamic acid per polypeptide; Dimension 17: Percentage of Phenylalanine per polypeptide; Dimension 18: Percentage of Glycine per polypeptide; Dimension 19: Percentage of Histidine per polypeptide; Dimension 20: Percentage of Isoleucine per polypeptide; Dimension 21: Percentage of Asparagine per polypeptide; Dimension 22: Percentage of Glutamine per polypeptide; Dimension 23: Percentage of Arginine per polypeptide; Dimension 24: Percentage of Serine per polypeptide; Dimension 25: Percentage of Threonine per polypeptide; Dimension 26: Percentage of non-canonical amino acid per polypeptide; Dimension 27: Percentage of Valine per polypeptide; Dimension 28: Percentage of Tryptophane per polypeptide; Dimension 29: Percentage of Tyrosine per polypeptide; Dimension 30: Cysteine content; Dimension 31: Percentage of coiled secondary structure per polypeptide; Dimension 32: Percentage of helical secondary structure per polypeptide; Dimension 33: Percentage of random secondary structure per polypeptide; Dimension 34: Score for structure around N-terminal cleavage site; Dimension 35: Score for structure around C-terminal cleavage site; Dimension 36: Number of helical blocks per polypeptide; Dimension 37: Isoelectric point of polypeptide; Dimension 38: Average molecular weight of polypeptide; Dimension 39: Sum of Van-der-Waals forces of each amino acid within polypeptide; Dimension 40: Sum of hydrophobicity values of each amino acid within polypeptide; Dimension 41-48: Mean values calculated based on principle component score vectors of hydrophobic, steric, and electronic properties per polypeptide; Dimension 49: Length of polypeptide.

In a preferred embodiment of the method of the present invention, protein sequences from step b) are only naturally occurring protein sequences found in the human secretome.

In another preferred embodiment, bioactive peptides are bioactive peptide hormones derived from precursor hormones.

Another subject-matter of the present invention refers to a bioactive peptide selected from the human secretome by using the method of the present invention.

In a preferred embodiment, the bioactive peptide is a bioactive peptide hormone. In a more preferred embodiment, the bioactive peptide hormone derives from a precursor protein.

In another preferred embodiment, the bioactive peptide has a sequence selected from the group consisting of the amino acid sequences of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38. 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138. 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185.

The invention pertains further to a peptide library comprising bioactive peptides identified through the method of the present invention.

In a preferred embodiment, the peptide library comprises bioactive peptides having a sequence selected from the group consisting of the amino acid sequences of SEQ ID NOS 1-185 cited above.

In a more preferred embodiment, the peptide library comprises bioactive peptide hormones.

In another more preferred embodiment, the peptide library comprises bioactive peptide hormones derived from precursor proteins.

Another subject-matter of the present invention refers to a computational device configured to identify bioactive peptides by using a binary support vector machine (SVM) based method, wherein:
a) a SVM algorithm is trained to learn to distinguish between bioactive and non bioactive peptides, said training comprising the steps of:
   a₁) generating vectors with 49 dimensions, each dimension resulting from the calculation of a molecular descriptor value, for a set of labelled known bioactive and labelled known non bioactive peptides, where the labels indicate whether the peptide is, respectively, bioactive or non bioactive;
   a₂) transferring the vector data generated in step a₁) to the SVM based algorithm, said algorithm calculating the optimal hyperplane that separates the vectors corresponding to the bioactive peptides and the non bioactive peptides, respectively;
b) protein sequences are provided from a publicly available human protein database;
c) secondary structure and cleavage sites within a protein sequence provided in step b) are predicted using computational methods; a set of 7 molecular descriptors is calculated based on said prediction step resulting in the generation of peptide fragments;
d) a set of 42 molecular descriptors corresponding to the physico-chemical properties of the peptide fragments generated in step c) is calculated;
e) the calculated values from step c) are transformed into scaled values between 0 and 1 to generate dimensions 1 to 7 of a 49-dimension-vector for each peptide fragment and the calculated values from step d) are transformed into scaled values between 0 and 1 to generate dimensions 8 to 49 of said vector for each peptide fragment;
f) the vectors generated in the step e) are presented to the trained SVM algorithm from step a) to measure the distance of each vector to the hyperplane calculated in step a₂); and
g) each peptide fragment is classified as bioactive peptide or non bioactive peptide, according to the distance measured in step f).

The invention pertains further to the use of the method of the present invention for the identification of therapeutic polypeptides, targets for drug intervention, ligands to discover relevant targets or biomarkers to monitor diseases.

The invention pertains further to the use of the peptide library of the present invention in a screening approach to interrogate intracellular signalling pathways; to create reagents to further the understanding of a pathway; to create novel forms of therapies and to identify pharmaceutically active compounds, targets for drug intervention, ligands to discover relevant targets or biomarkers to monitor diseases.

The invention is also directed to a pharmaceutical composition comprising a bioactive peptide having a sequence selected from the group consisting of the amino acid sequences of SEQ ID NOS 1-185 as bioactive agent.

### DETAILED DESCRIPTION OF THE INVENTION

The present invention is directed to novel bioactive polypeptides and to an in silico method to identify such bioactive polypeptides.

In the present invention, a polypeptide is considered as bioactive if it has an interaction with or an effect on any cell tissue in the human body. Bioactive peptides have the potential to be used as therapeutic polypeptides, targets for drug intervention, ligands to discover relevant targets (eg. GPCR deorphaning) or biomarkers to monitor diseases. Bioactive peptides include, among others, bioactive peptide hormones. Peptide hormones are characterized by their high specificity as well as their effectiveness in very low concentrations. Peptide hormones are initially synthesized as larger precursors, or prohormones.

A precursor is a substance from which another usually more active or mature substance is formed. A protein precursor is an inactive protein (or peptide) that can be turned into an active form by post-translational modification. Several cleavage sites are involved in the modification of the precursor to produce the mature protein: signal sequence cleavage sites, protease cleavage sites, amidation sites, etc.

The name of the precursor for a protein is often prefixed by pro or pre. Precursors are often used by an organism when the subsequent protein is potentially harmful, but needs to be available on short notice and/or in large quantities.

The terms “polypeptide”, “peptide” and “protein” are used interchangeably herein to refer to a polymer consisting of amino acid residues linked by covalent bonds. These terms include parts or fragments of full length proteins, such as, for example, peptides, oligopeptides and shorter peptide sequences consisting of at least 2 amino acids, more particularly peptide sequences consisting of 4-45 amino acids.

In addition, these terms include polymers of modified amino acids, including amino acids which have been post-translationally modified, for example by chemical modification including but not restricted to amidation, glycosylation, phosphorylation, acetylation and/or sulphation reactions that effectively alter the basic peptide backbone. Accordingly, a polypeptide may be derived from a naturally-occurring protein, and in particular may be derived from a full-length protein by chemical or enzymatic cleavage, using reagents such as CNBr, or proteases such as trypsin or chymotrypsin, amongst others. Alternatively, such polypeptides may be derived by chemical synthesis using well known peptide synthetic methods.

An amino acid is any molecule that contains both amine and carboxylic acid functional groups. Amino acid residue is what is left of an amino acid once a molecule of water has been lost (an H+ from the nitrogenous side and an OH− from the carboxylic side) in the formation of a peptide bond, the chemical bond that links the amino acid monomers in a protein chain.

Each protein has its own unique amino acid sequence that is known as its primary structure. Primary structure is fairly straightforward and refers to the number and sequence of amino acids in the protein or polypeptide chain. The covalent peptide bond is the only type of bonding involved at this level of protein structure. The sequence of amino acids in a protein is dictated by genetic information in DNA, which is transcribed into RNA, which is then translated into protein. So protein structure is genetically determined.

The next level of protein structure generally refers to the amount of structural regularity or shape that the polypeptide chain adopts. A natural polypeptide chain will spontaneously fold into a regular and defined shape. Two main types of secondary structure have been found in proteins namely a-helix, and b-pleated sheet.

The tertiary structure of a polypeptide chain is the next level of conformation or shape adopted by the alpha-helices or beta-pleated sheets of the chain. Most proteins tend to fold into shapes that are broadly classified as globular in arrangement, and some, particularly structural proteins form long fibres. These are the main forms of gross tertiary structure. A term often used is domain, which refers to a compact unit of globular structure in a polypeptide chain.

The unique shape of each protein determines its function in the body.

Also included within the scope of the definition of a “polypeptide” are amino acid sequence variants. These may contain one or more preferably conservative, amino acid substitutions, deletions, or insertions, in a naturally-occurring amino acid sequence which do not alter at least one essential property of said polypeptide, such as, for example, its biological activity. Such polypeptides may be synthesized by chemical polypeptide synthesis. Conservative amino acid substitutions are well-known in the art. For example, one or more amino acid residues of a native protein can be substituted conservatively with an amino acid residue of similar charge, size or polarity, with the resulting polypeptide retaining functional ability as described herein. Rules for making such substitutions are well known.

More specifically, conservative amino acid substitutions are those that generally take place within a family of amino acids that are related in their side chains.

Genetically-encoded amino acids are generally divided into four groups: (1) acidic=aspartate, glutamate; (2) basic=lysine, arginine, and histidine; (3) non-polar=alanine, valine, leucine, isoleucine, proline, phenylalanine, methionine, and tryptophan; and (4) unchargedpolar=glycine, asparagine, glutamine, cysteine, serine, threonin, and tyrosine. Phenylalanine, tyrosine and tryptophan are also jointly classified as aromatic amino acids. One or more replacements within any particular group such as, for example, the substitution of leucine for isoleucine or valine are alternatively, the substitution of aspartate for glutamate or threonin for serine, or of any other amino acid residue with a structurally-related amino acid residue will generally have an insignificant effect on the function of the resulting polypeptide.

Included in the scope of the definition of the term “polypeptide” is a peptide whose biological activity is predictable as a result of its amino acid sequence corresponding to a functional domain. Also encompassed by the term “polypeptide” is a peptide whose biological activity could not have been predicted by the analysis of its amino acid sequence.

In the present invention, a support vector machine algorithm (SVM) is used to distinguish between polypeptides that have an activity in vivo and polypeptides that do not have an activity in vivo.

Support Vector Machine (SVM):

A Support Vector Machine (SVM) is a universal learning machine that, during a training phase, determines a decision surface or “hyperplane”. The decision hyperplane is determined by a set of support vectors selected from a training population of vectors and by a set of corresponding multipliers. The decision hyperplane is also characterised by a kernel function.

The mathematical basis of a SVM is explained in the book by John Shawe Taylor & Nello Cristianini—Cambridge University Press, 2000, entitled “Support Vector Machines and other kernel-based learning methods” and in an article by Chih-Chung Chang and Chih-Jen Lin entitled “LIBSVM—A Library for Support Vector Machines”, 2001.

Subsequent to the training phase, a SVM operates in a testing phase during which it is used to classify test vectors on the basis of the decision hyperplane previously determined during the training phase (Noble, 2006).

Support Vector Machines find application in many and varied fields. For example, in a paper by H. Kim and H. Park entitled “Prediction of protein relative solvent accessibility with support vector machines and long-range interaction 3d local descriptor”, SVMs are applied to the problem of predicting high resolution 3D structure in order to study the docking of macro-molecules.

In the present invention, a support vector machine algorithm (SVM) is used to distinguish between polypeptides that have an activity in vivo and polypeptides that do not have an activity in vivo.

From a practical point of view, a SVM is implemented by means of a computational device such as a personal computer in the present invention.

The computational device includes one or more processors that execute a succession of different softwares, as described in the exemplification section (1.1.), containing instructions for implementing a method according to the present invention.

Training of SVM and Model Generation:

In order to train the SVM model, vectors with 49 dimensions were generated using the program routine described in the experimentation section (1.1.) and schematically shown in FIG. 1.

For the SVM training set, information on known bioactive peptides can be extracted from any public available human protein database, such as Swissprot. Preferably bioactive peptides with a length between 4 and 55 amino acids were extracted from their precursor according to their annotation in Swissprot and labeled as positive examples used for training of the SVM algorithm. All other fragments generated with a length between 4-55 amino acids from the same known peptide hormone precursors that have no assigned function were used as negative trainings set for SVM training. As the SVM is a binary system, bioactive peptides were labeled as +1 and non bioactive peptides were labeled as −1.

Similarly, bioactive and non bioactive peptides with a length between 56 and 300 amino acids were used to train a second model to predict longer polypeptides. In order not to over-represent negative examples, the final SVM training sets for short (4-55 amino acids) and long (56-300 amino acids) respectively were adjusted to an equal number of positive and negative training data by randomly selecting the same number of negatives from all negative peptides.

To transform the information hidden in the bioactive and non bioactive peptides, a set of 49 descriptors was defined and used for training of a SVM. The performance of a SVM model strongly depends on the quality of the chosen descriptors used to describe the peptides. In the present invention, the first 7 descriptors reflect the likelihood of a polypeptide to be produced by the human body. These 7 dimensions were calculated by employing a set of protease prediction site tools to the peptide hormone precursor sequence (FIG. 1). The resulting scores of each program output were directly used as descriptors. The remaining 42 dimensions reflect important physico-chemical properties of each generated fragment (i.e. a bioactive or a non bioactive peptide). The 49 descriptors used in the present invention are listed in the point 3 of the exemplification section.

To each peptide corresponds a unique combination of 49 descriptors. The different peptides can be represented as points in a multidimensional space where each dimension corresponds to one of the descriptors. The SVM seeks to find a boundary that best separates the two sets of points corresponding to the bioactive and the non bioactive peptides. This boundary is called the optimal hyperplane that best separates the two classes of objects in an n-dimensional space, namely the vectors corresponding to the bioactive peptides and the non bioactive peptides, respectively.

The resulting SVM models learn to distinguish between bioactive and non bioactive peptides. The best model is chosen which has the highest performance based on the ranking of an independent test set of bioactive and non bioactive peptides. To test the models, the performance of all generated models was tested and the two best models for short peptides (4-55 amino acids) and longer polypeptides (56-300 amino acids) were chosen, respectively.

Identification of Bioactive Peptides:

After training, the resulting trained SVM model is able to identify bioactive peptides for which no bioactivity had been characterized.

A schematic overview of the method disclosed in the invention is given in FIG. 1 to explain the steps involved in peptide library generation. As input value a protein sequence provided from a publicly available human protein database, such as Swissprot, is used. In step 1, all potential protease cleavage sites are predicted using a set of tools to predict these events. The respective cleavage site positions are saved for each precursor sequence. In addition, the secondary structure is deduced for the entire protein precursor sequence. Based on the predicted cleavage sites within the precursor sequence, all potential fragments are generated (step 2) and are used as input for step 3.

Step 3 comprises the calculation of physico-chemical properties of each peptide fragment (list in point 3 of the exemplification section). In general, information on the amino acid frequency within each fragment, the secondary structure of each fragment, the isoelectric point of each fragment, average molecular mass of each fragment, hydrophobicity of each fragment, the sum of all van-der-Waals forces for each amino acid within the fragment, the sum of all commonly used amino acid descriptors (i.e. VHSE value for each amino acid based on Mei et al., 2005) for each amino acid within the fragment and the fragment length are taken into account to transform the biological information into numerical values.

Calculated values from step 1 and 3 are transformed in steps 4a and 4b to give scaled values between 0 and 1, respectively, to generate a 49 dimensional vector for each fragment.

In step 5 the vectors are presented to the trained SVM model to measure the distance of each vector to the hyperplane. The SVM output is then used in step 6 to decide whether the peptide is likely to be bioactive or not. 49-dimensional vectors corresponding to the bioactive peptides identified through the method of the present invention are listed in FIG. 3.

In order to significantly reduce the potential number of structures in a peptide library, in the present invention only naturally occurring protein sequences found in the human secretome were used as primary structures to generate peptide libraries. The human secretome is the whole information encoded in the DNA that corresponds to all human proteins that are secreted by the cells.

Potentially secreted human proteins which were used as precursor sequences to find novel bioactive peptides were extracted from the publicly available sequence databases listed in point 1.1.of the exemplification section.

Distinct parts of the primary sequences of secreted proteins, i.e. protein precursors, were used as templates to deduce novel bioactive peptides. The peptide length was restricted to 4-45 amino acids to render the peptides amenable to chemical synthesis.

Subsequent to the identification of novel bioactive peptides through the method of the present invention, antimicrobial assays were performed to test the bioactivity of the latter peptides. These assays are detailed in point 6 of the exemplification section.

The present invention further relates to a peptide library comprising bioactive peptides identified through the SVM model method described above. The amino acid sequences of the 185 bioactive peptides identified through the method of the present invention and comprised in the peptide library of the present invention are listed in FIG. 2.

A peptide library is a newly developed technique for protein related study. A peptide library contains a great number of peptides that have a systematic combination of amino acids. Usually, peptide libraries are synthesized on solid phase, mostly on resin, which can be made as flat surface or beads. A peptide library provides a powerful tool for drug design, protein-protein interactions, and other biochemical as well as pharmaceutical applications. The peptide library of the present invention can be used in a screening approach to interrogate intracellular signalling pathways, to create reagents to further the understanding of a pathway, to create novel forms of therapies and to identify pharmaceutically active compounds, targets for drug intervention, ligands to discover relevant targets or biomarkers to monitor diseases.

The polypeptides of the present invention have hormonal activity. Therefore, the polypeptides of the invention are useful as drugs, for example therapeutic polypeptides, ligands to discover relevant targets (e.g. GPCRs), targets for drug intervention (e.g. targets for monoclonal antibodies, receptor fragments), biomarkers to monitor diseases (in combination with tool antibodies to detect peptide fragments in body fluids), protein kinase inhibitors and substrates, T-cell epitopes, peptide mimotopes of receptor binding sites, etc.

The DNAs coding for the peptide or precursor of the invention are useful, for example, as agents for the gene therapy, treatment or prevention of cardio-vascular diseases, hormone-producing tumours, diabetes, gastric ulcer and the like, hormone secretion inhibitors, tumour growth inhibitors, neural activity etc. Furthermore, the DNAs of the invention are useful as agents for the gene diagnosis of diseases such as cardio vascular disease, hormone-producing tumours, diabetes, gastric ulcer and the like.

Exemplification

The invention now being generally described will be more readily understood in reference to the following examples, which are included merely for purposes of illustration of certain aspects and embodiments of the present invention and are not intended to limit the invention.

1. Databases and Computer Programs

1.1. Databases

The following publicly available sequence databases were used to extract potentially secreted human proteins which were used as precursor sequences to find novel bio-active peptides:

Human genome (NCBI 33 assembly, 1 Jul. 2003) translated into protein, subset; International Protein Index, Swissprot (Release 50.3 of 11 Jul. 2006) and TrEMBL (Releases: August 2003-March 2006);

For training of SVM based algorithms, information on known bioactive peptides was extracted from Swissprot.

1.2. Computer Programs

1.1 Signal P Version 2.0 (Nielsen et al., 1997)

Objective: This program was used to detect potential signal sequences and determine the potential human secretome. It was used with a cut off score of 0.98. Signal P version 2.0 predicts the presence and location of signal peptide cleavage sites in amino acid sequences from different organisms: The method incorporates a prediction of cleavage sites and a signal peptide/non-signal peptide prediction based on a combination of several artificial neural networks and hidden Markov models.

1.2 ProP Version 1.0 (Duckert et al., 2004)

Objective: This program was used to detect potential cleavage sites in protein sequences.

The cut off score used was set to 0.11. This program predicts arginine and lysine propeptide cleavage sites in eukaryotic protein sequences using an ensemble of neural networks. Furin-specific prediction is the default. It is also possible to perform a general proprotein convertase (PC) prediction.

1.3. Amidation Site Prediction and Prediction of Protease Cleavage Sites (Rohrer, 2004)

Objective: The program Hamid predicts amidation sites in protein sequences. The program Hmcut predicts protease cleavage sites in protein sequences that take place before a basic amino acid residue (Lys, Arg). Both programs are based on Hidden Markov Models and utilize the software version Hmmer 2.3.2 (Durbin et al. 1998).

1.4 Support Vector Machine (Chang and Lin, 2001)

LIBSVM is an integrated software for support vector classification, (C-SVC, nu-SVC), regression (epsilon-SVR, nu-SVR) and distribution estimation (one-class SVM). The following SVM specifications were used: SVM_type: nu-SVC; Kernel_type: radial basis function.

1.5. PsiPred Version 2.45 (Jones, 1999)

Method for protein secondary structure prediction. The method was used as described in Jones, 1999.

1.6. Calculation of Isoelectric Points

Objective: Caluclation of isoelectric points of polypeptides. This was done according to Gasteiger et al. 2005.

1.7. Perl—Practical Extraction and Report Language

Objective: Perl is a dynamic programming language created by Larry Wall and first released in 1987.

2. Training of SVM

For the supervised learning process, known bioactive polypeptide precusors were extracted from commonly used public databases such as Swissprot using the following SRS (Sequence Retrieval System on www.expasy.org) query statement: Organism=vertebrate; Sequence_length=30:300; Feature_key=signal; Keywords=cytokine or hormone or bombesin or bradykinin or glucagon or growth factor or insulin or neuropeptide or opioid peptide or tachykinin or thyroid hormone or vasoconstrictor or vasodilator. This query yields a set of known peptide hormone precursors in which their bioactive peptides are readily available by the annotation of the Swissprot database. Therefore these sequences can be used to deduce a set of bioactive and non bioactive peptides for training of an SVM based model.

3. Molecular Descriptors Used to Build the Vectors

The performance of a SVM model strongly depends on the quality of the chosen descriptors used to describe the peptides.

In the present invention, the following descriptors were chosen:

Dimension 1-7 represent the likelihood of a polypeptide to be produced in the human body and was calculated by a combination of different protease cleavage site prediction tools. The results of these tools represent in the first 7 dimensions of the vector.

Dimension 1: N-terminal ProP score;

Dimension 2: N-terminal Hmcut score;

Dimension 3: N-terminal fragment (fixed value of 0.2)

Dimension 4: C-terminal ProP score;

Dimension 5: C-terminal Hmcut score;

Dimension 6: C-terminal Hamid score;

Dimension 7: C-terminal fragment (fixed value of 0.2)

Physico-chemical properties of the polypeptides were calculated and represent the following 42 dimensions of the vector.

Dimension 8: Percentage of acidic amino acids (E, N, Q) per polypeptide

Dimension 9: Percentage of positively charged amino acids (R, H) per polypeptide

Dimension 10: Percentage of aromatic amino acids (F, Y, W) per polypeptide

Dimension 11: Percentage of aliphatic amino acids (G, V, A, I) per polypeptide

Dimension 12: Percentage of Proline per polypeptide

Dimension 13: Percentage of reactive amino acids (S, T) per polypeptide

Dimension 14: Percentage of Alanine per polypeptide

Dimension 15: Percentage of Cysteine per polypeptide

Dimension 16: Percentage of Glutamic acid per polypeptide

Dimension 17: Percentage of Phenylalanine per polypeptide

Dimension 18: Percentage of Glycine per polypeptide

Dimension 19: Percentage of Histidine per polypeptide

Dimension 20: Percentage of Isoleucine per polypeptide

Dimension 21: Percentage of Asparagine per polypeptide

Dimension 22: Percentage of Glutamine per polypeptide

Dimension 23: Percentage of Arginine per polypeptide

Dimension 24: Percentage of Serine per polypeptide

Dimension 25: Percentage of Threonine per polypeptide

Dimension 26: Percentage of non-canonical amino acid (undefined) per polypeptide

(Please not that this dimension does not contain any value other than 0 as input)

Dimension 27: Percentage of Valine per polypeptide

Dimension 28: Percentage of Tryptophane per polypeptide

Dimension 29: Percentage of Tyrosine per polypeptide

Dimension 30: Cysteine content (zero, even or odd number set to 0.5, 1 or 0, respectively

Dimension 31: Percentage of coiled secondary structure per polypeptide

Dimension 32: Percentage of helical secondary structure per polypeptide

Dimension 33: Percentage of random secondary structure per polypeptide

Dimension 34: Score for structure around N-terminal cleavage site

Dimension 35: Score for structure around C-terminal cleavage site

Dimension 36: Number of helical blocks per polypeptide

Dimension 37: Isoelectric point of polypeptide

Dimension 38: Average molecular weight of polypeptide

Dimension 39: Sum of Van-der-Waals forces of each amino acid within polypeptide

Dimension 40: Sum of hydrophobicity values of each amino acid within polypeptide

Dimension 41-48: Mean values calculated based on principle component score vectors of hydrophobic, steric, and electronic properties per polypeptide (Mei et al. 2005)

Dimension 49: Length of polypeptide

Wherever applicable, the values for dimension 1-49 were scaled to be in the range between 0 and 1.

The input vectors for training and prediction contain 49 dimensions, however in the current format only 48 are utilized since dimension 26 is set to zero for all fragments (percentage of non-cannonical amino acids per fragment). This is due to the lack of appropriate training data containing non-cannonical amino acids, but can be included in future models.

4. Testing of the Models

The best model is chosen which has the highest performance based on the ranking of an independent test set of bioactive and non bioactive peptides. To test the models, the performance of all generated models was tested and the two best models for short peptides (4-55 amino acids) and longer polypeptides (56-300 amino acids) were chosen, respectively.

As a result, an overall prediction accuracy of 90.7% for short peptides and 94% for longer peptides was achieved. Using an independent test set the disclosed method correctly identifies around 93% of bio-active peptides and around 91% of the non-active peptides.

5. Identification of Bioactive Peptides

During the ranking step (Step 6, FIG. 1), the highest scoring peptides per precursor are chosen that have a length shorter than 46 amino acids. In this ranking process, all fragments that have after SVM classification a distance greater than |0.65| and are localized with the negative training data set (i.e. a score of −0.65 or lower) are readily discarded even if they represent the highest scoring peptides per protein precursor.

6. Antimicrobial Assays to Test the Bioactivity of the Peptides Identified Through the Method of the Present Invention

6.1. Assay Technology

The micro dilution test represents a homogenous method for determining the number of viable bacterial or yeast cells in culture. It relies on the fact that living bacteria or yeast are turbid in culture. Turbidity can be measured as light absorbance with a photometer and is correlated with the number of cells in the sample.

6.2. Materials and Methods

Bacterial and Yeast Strains

The strains used in the course of the experiments are Escherichia coli (E. coli ATCC 25922), Staphylococcus aureus (S. aureus ATCC 29213) and Candida albicans (C. albicans FH 2173).

Pre-Cultivation of All Test Strains

The cultivation of the strain starts with building up a cryostock that can be used for multiple inoculations of pre-cultures.
1. Streak the bacteria onto the surface of a Mueller Hilton (MH)-agar plate by using an inoculation loop, and incubate the agar plate for 3 days at 37° C. For yeast use the same procedure but with Sabouraud dextrose (SD)-agar.
2. Inoculate a 100 ml shaking flask containing 30 ml MH broth with one loop of bacteria and incubate the flask for 1 day at 37° C. and 180 rpm. For yeast apply the same conditions in SD broth.
3. Remove the hypertonic cryo-preservative solution from the Cryobank (CRYO/G) plastic vials, each containing 25 green glass beads, by using a sterile pipette.
4. Fill each vial with 2 ml of the bacterial/yeast suspension, close the vial, and mix carefully.
5. Remove as much of the bacterial/yeast culture supernatant from the vial as possible. The surface of the beads is now covered with bacteria/yeast. The amount of liquid remaining in the vial should be as low as possible to prevent clumping of the beads. One bead is used for the inoculation of one pre-culture (30 ml MH/SD broth in a 100 ml shaking flask).
6. Store the Cryobank (CRYO/G) vials at −80° C.
7. Quality/sterility check: Remove a Cryobank (CRYO/G) vial from the freezer and place it into a Cryoblock (CRYO/Z). Open the vial, remove one bead and immediately streak the bead over the surface of a MH/SBD agar plate. Incubate the plate for 3 days at 37° C. Verify that only the test strain has grown by examining the colony morphology.

Preparation of Test Culture Using MH Broth

The test strain vial is removed form the Cryobank. One bead is removed with a sterile pipette and inoculated in a 100 ml Erlenmeyer with 30 ml MH and SD broth for bacteria and yeast, respectively. Grow the culture for 18 h at 37° C. and 180 rpm. The optical density is adjusted with MH broth to a cell density corresponding to 10⁸ cells/ml for all test strains. The standard inoculum culture for the assay is diluted 1:100 to the final concentration of 10⁶ CFU/ml (colony forming units/ml).

Peptide Dilutions

The compounds are diluted serially (10 dilution steps) from the standard initial concentration of 125 μM to a final concentration of 0.24 μM. The initial DMSO concentration is 1.4% in all samples and controls.

Standard Antibiotic Dilutions for Dose Response Curves

For dose response experiments dilute the compounds serially (16 dilution steps) with MH broth. Final compound concentrations range between 64 μg/ml and 0.002 μg/ml. The initial DMSO concentration is 1.4% in all samples and controls.

| | Supplier | Cat No | Function |
|---|---|---|---|
| Mueller Hinton (MH) | Becton Dickinson | 275730 | Culture medium |
| broth | | | |
| Sabouraud dextrose | Becton Dickinson | 238230 | Culture medium |
| (SD) broth | | | |
| DMSO | Merck | 102 931 | Solvent |
| Nystatin | Calbiochem | 475914 | Antibiotics |
| Cyprobay 100 | Bayer | | |
| Greiner, 384 | Greiner | 781182 | Assay Plates |
| SPECTRAFluor Plus | Tecan | — | Reader Absorbance |

Assay Protocol
Pre-culture the bacteria in 30 ml MH broth at 37° C. for 18 h (100 ml Erlenmeyer flask)
Pre-culture the yeast in 30 ml SD broth at 37° C. for 18 h (100 ml Erlenmeyer flask)
Adjust the cell suspension with MH broth to 10⁶ CFU/ml (test culture)

Assay:
Add 10 μl compound in DMSO and 30 μl MH broth to the first vial
Transfer 20 μl from the first vial in the second that contains 20 μl MH broth
The last step is repeated 8 times (peptides, 10 dilution steps) or 14 times (antibiotics, 16 dilution steps)
Add 10 μl test culture suspension to each vial (10 vials for the peptides and 16 vials for the antibiotics)

| | |
|---|---|
| start cell inoculum | 5 × 10⁵ CFU |
| start DMSO concentration | 12.5% |
| start/final compound concentration | 125 μM-0.24 μM |
| start/final antibiotic concentration | 64 μg/ml-0.002 μg/ml |

Incubate at 37° C. for 18 h by 5% relative humidity and 5% CO₂
Read absorbance at 590 nm with 5 flashes

Controls:
High controls: MH broth with bacteria (growth control, high signal)
Low controls: MH broth without bacteria (sterile control, low signal)

6.3. Sensitivity Testing with Antibiotics

In order to evaluate the suitability of the assay for the identification of potential drugs, the dose dependent effects of a number of antibiotics were tested using the conditions described under ‘Materials and Methods’. Cyprofloxacin expected to be active against E. coli and S. aureus and Nystatin against C. albicans. The calculated IC50 values for these antibiotics are given in FIG. 4 in μg/ml.

6.4. Assay Results

The peptides were tested against the test strains E. coli (ATCC 25922), S. aureus (ATCC 29213) and C. albicans (FH 2173). The peptides A003500589 and A003500548 showed IC50 values of 7.25 μg/ml and 6.79 μg/ml, respectively, against E. coli. No activities were found against S. aureus and C. albicans.

### REFERENCES

Chih-Chung Chang and Chih-Jen Lin; “LIBSVM: a library for support vector machines”; 2001

Peter Duckert, Søren Brunak and Nikolaj Blom; “Prediction of proprotein convertase cleavage sites”; Protein Engineering, Design and Selection, 17:107-112, 2004

Durbin R, Eddy S, Krogh A and Mitchison G; “The theory behind profile HMMs: Biological sequence analysis: probabilistic models of proteins and nucleic acids”; Cambridge University Press, 1998.

C. Falciani, L. Lozzi, A. Pini, L. Bracci; “Bioactive Peptides from Libraries”; Chemistry & Biology, Volume 12, Issue 4, Pages 417-426, 2005

Gasteiger E., Hoogland C., Gattiker A., Duvaud S., Wilkins M. R., Appel R. D., Bairoch A.; “Protein Identification and Analysis Tools on the ExPASy Server”; (In) John M. Walker (ed): The Proteomics Protocols Handbook, Humana Press, 2005

Jones, D. T.; “Protein secondary structure prediction based on position-specific scoring matrices”; J. Mol. Biol. 292:195-202, 1999

H. Kim and H. Park; “Prediction of protein relative solvent accessibility with support vector machines and long-range interaction 3d local descriptor”; Proteins, 54(3): 557-62, 2004

Mei, H., Liao, T. H., Zhou, Y., and Li, S. Z.; “A new set of amino acid descriptors and its application in peptide QSARs”; Biopolymers Vol. 80, 775-786, 2005

Henrik Nielsen, Jacob Engelbrecht, Søren Brunak and Gunnar von Heijne; “Identification of prokaryotic and eukaryotic signal peptides and prediction of their cleavage sites”; Protein Engineering, 10:1-6, 1997

Noble W S.; “What is a support vector machine?”; Nat. Biotechnol. 24(12):1565-7, 2006

Rohrer, S.; “Prediction of post-translational processing sites in peptide hormone precursors”; Diplomarbeit, Universitat Würzburg, 2004

John Shawe Taylor & Nello Cristianini; “Support Vector Machines and other kernel-based learning methods”; Cambridge University Press, 2000

### DESCRIPTION OF THE FIGURES

FIG. 1:

A schematic overview of the method disclosed in the invention is given in FIG. 1 to explain the steps involved in peptide library generation.

FIG. 2:

FIG. 2 shows the amino acid sequences of the 185 bioactive peptides selected based on shared physico-chemical properties.

FIG. 3:

FIG. 3 shows the input vectors of the 185 peptides identified as bioactive by the trained SVM algorithm.

FIG. 4:

FIG. 4 shows the calculated IC50 values for antibiotics in μg/ml.

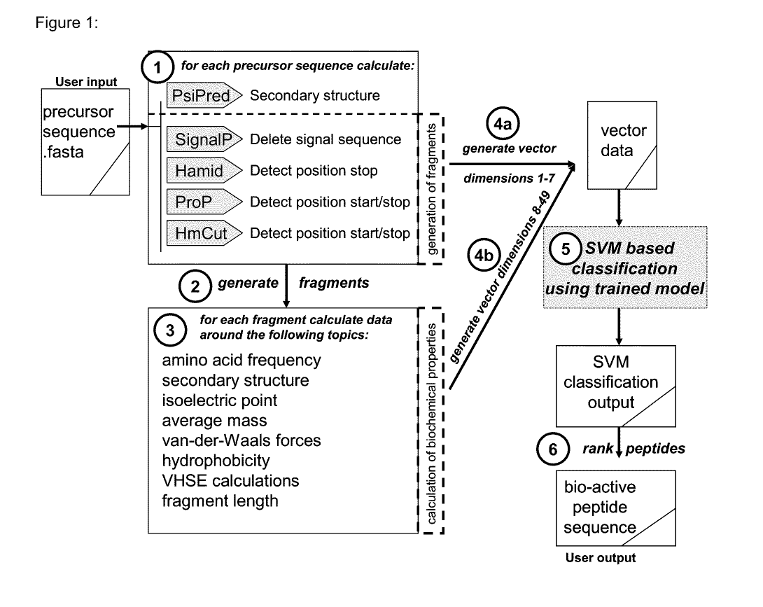

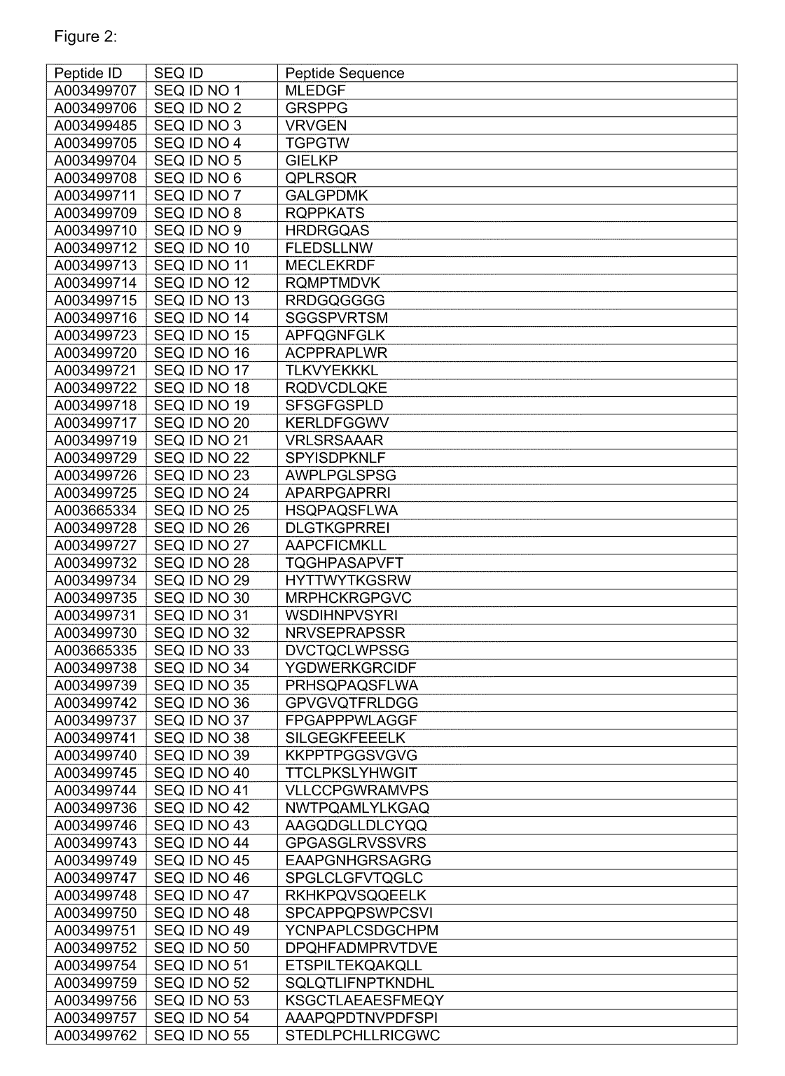

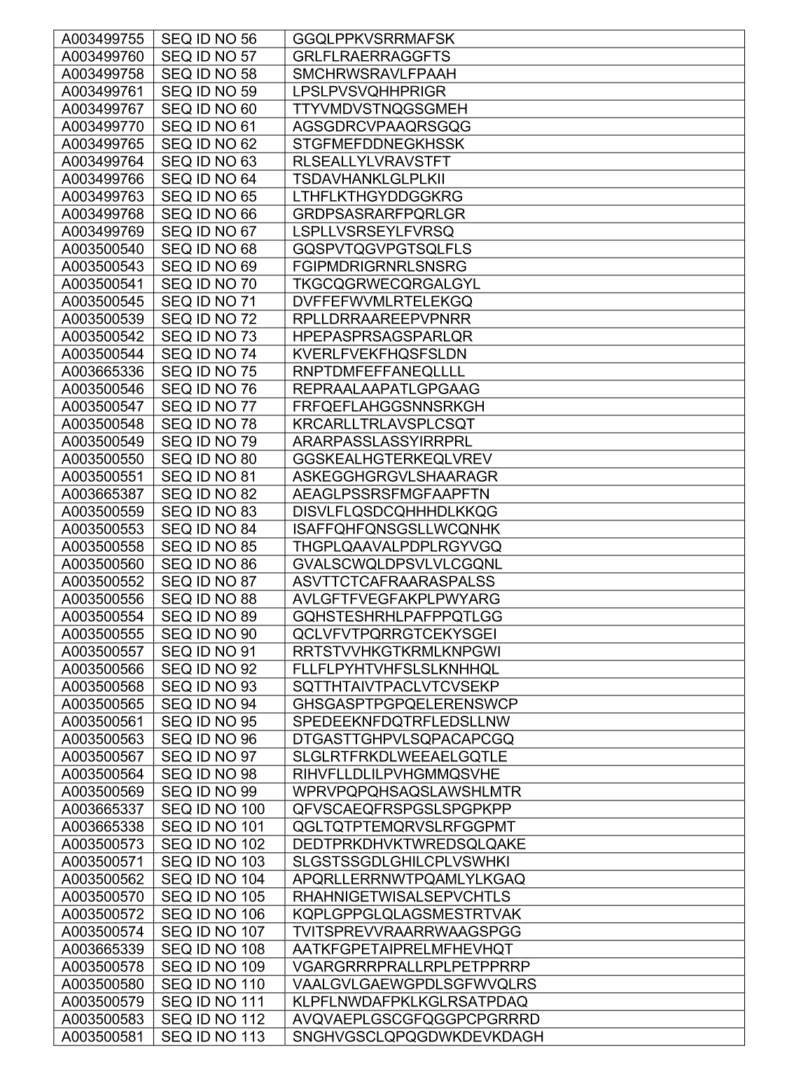

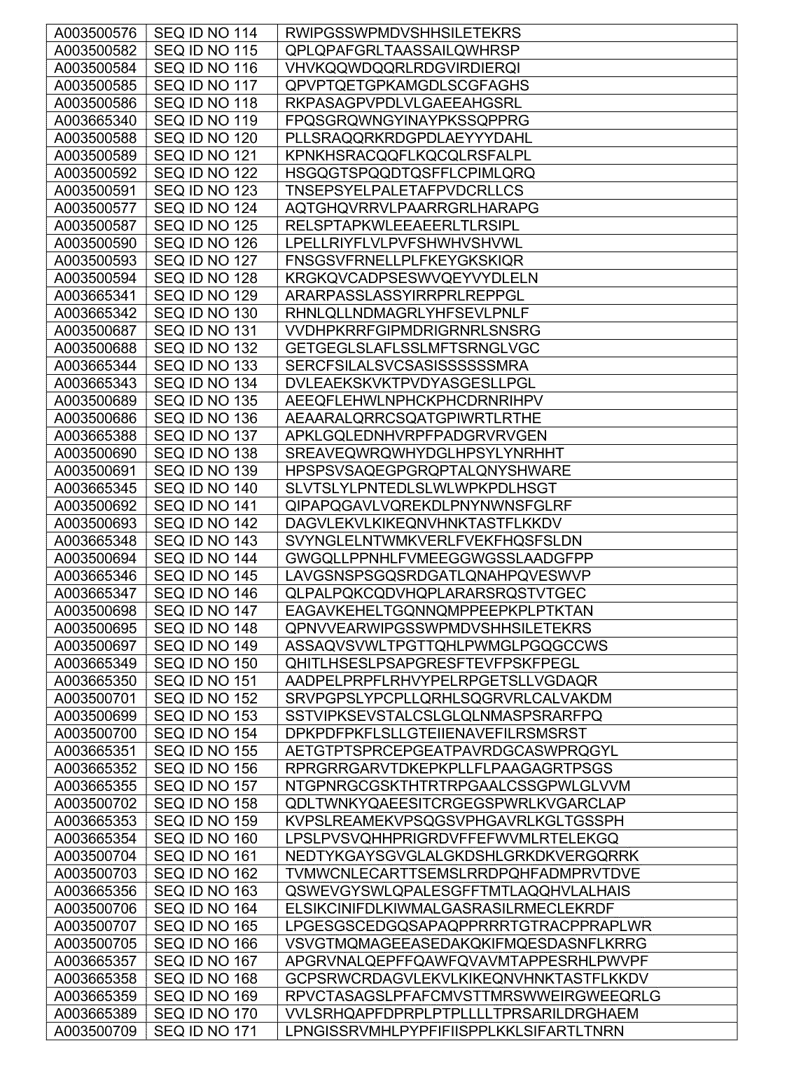

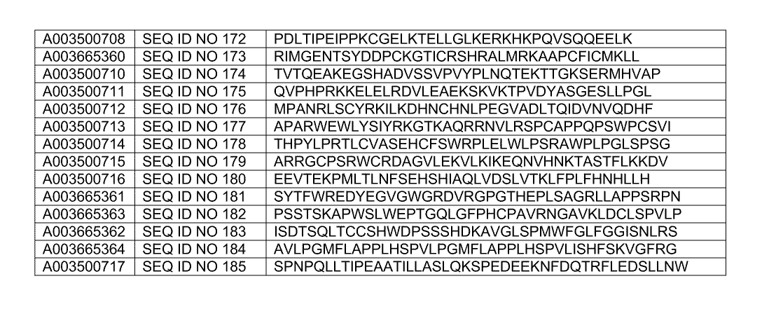

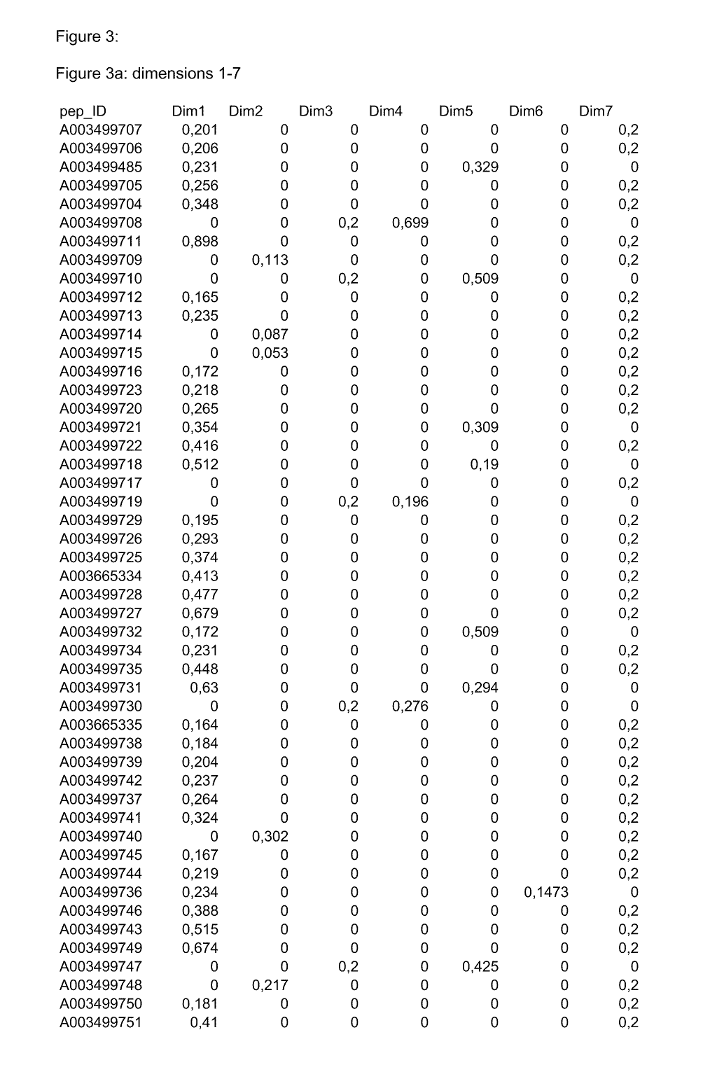

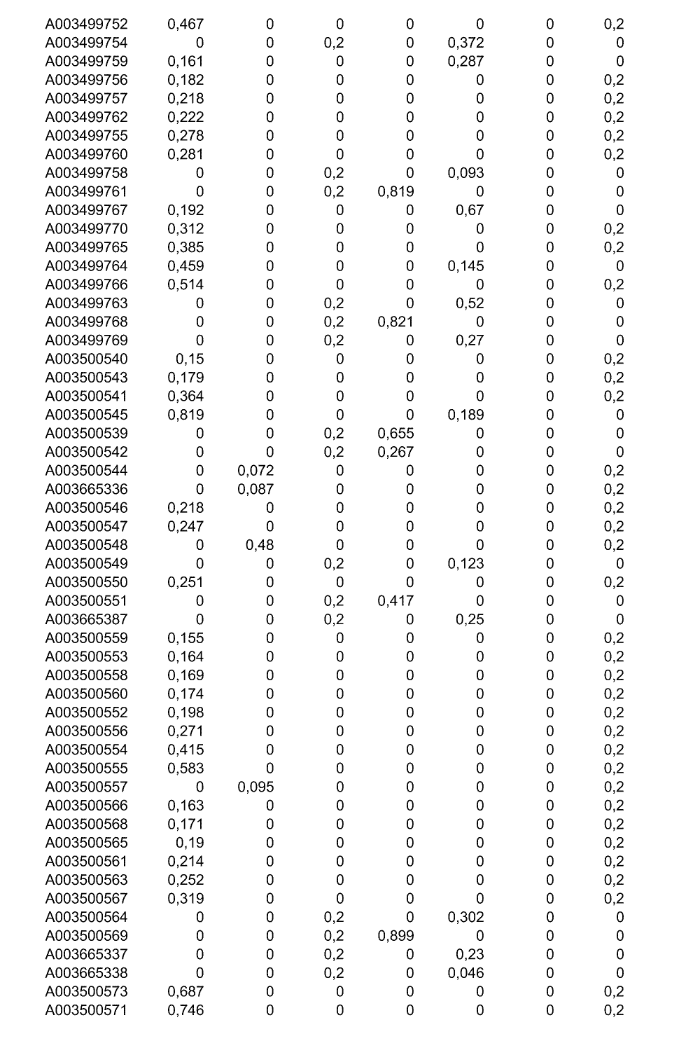

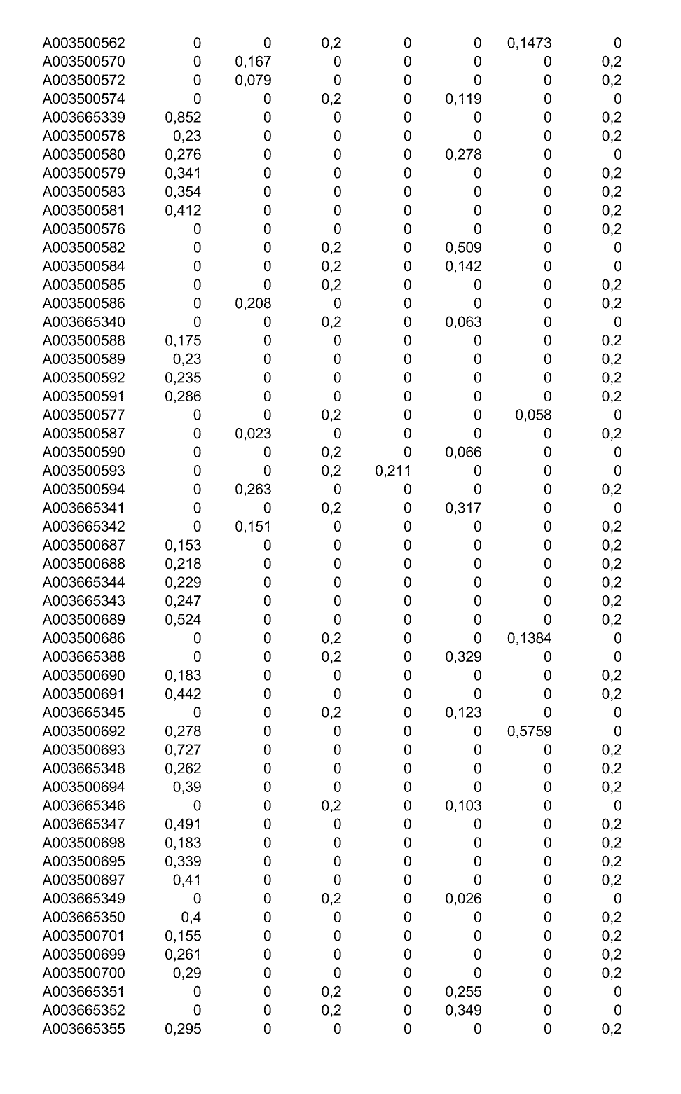

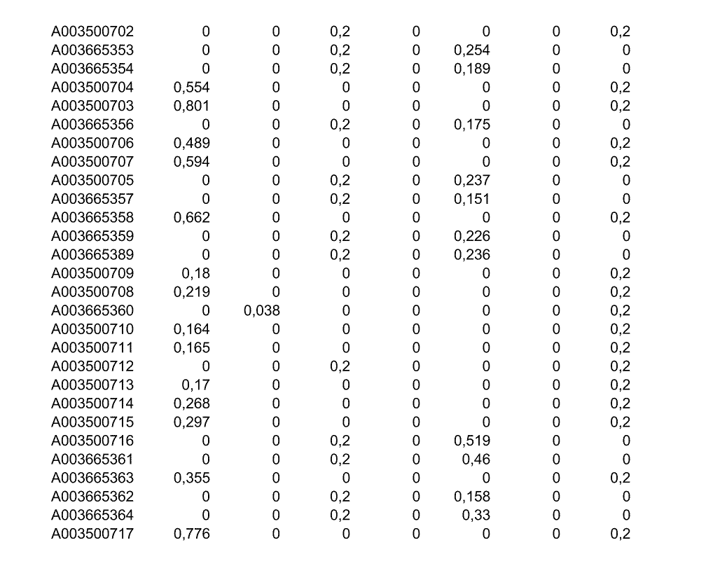

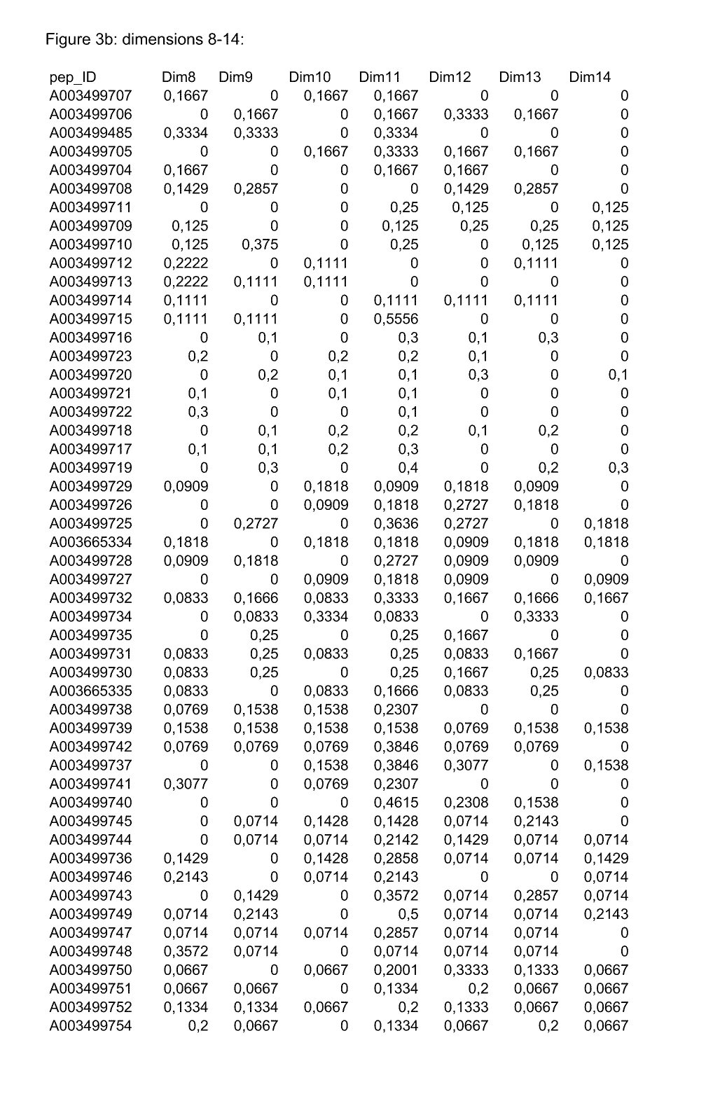

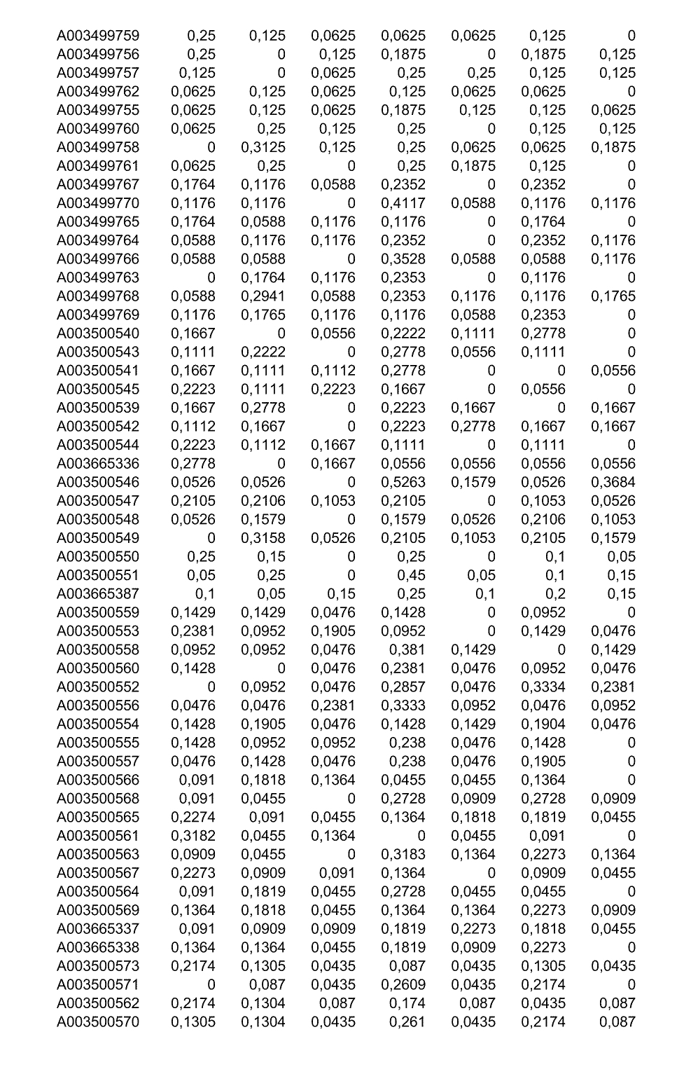

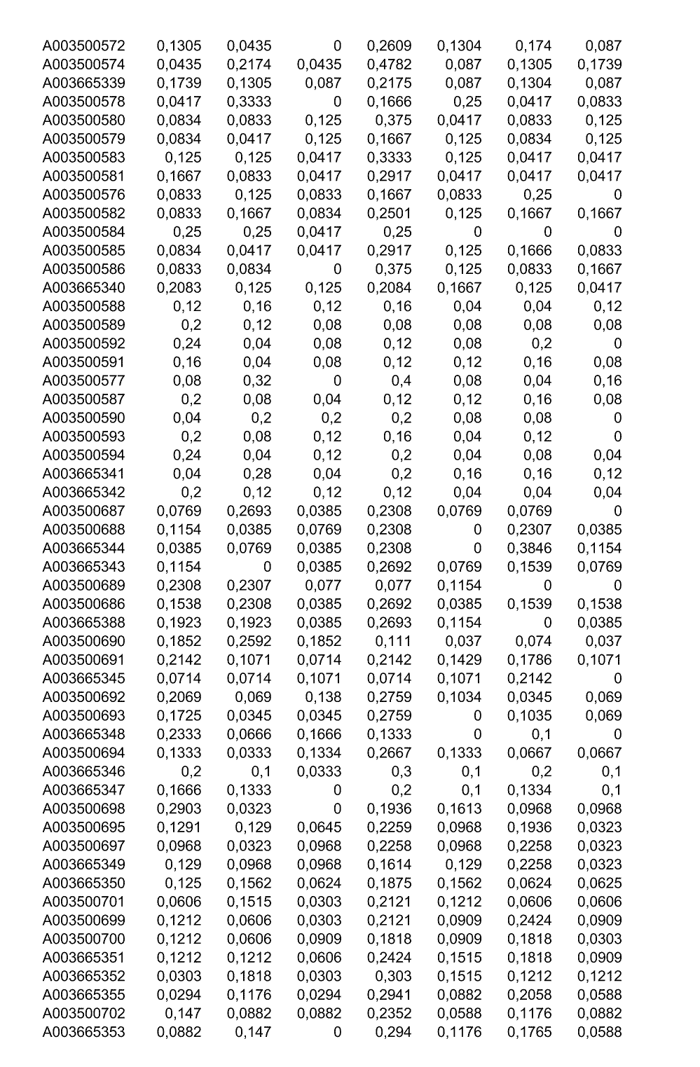

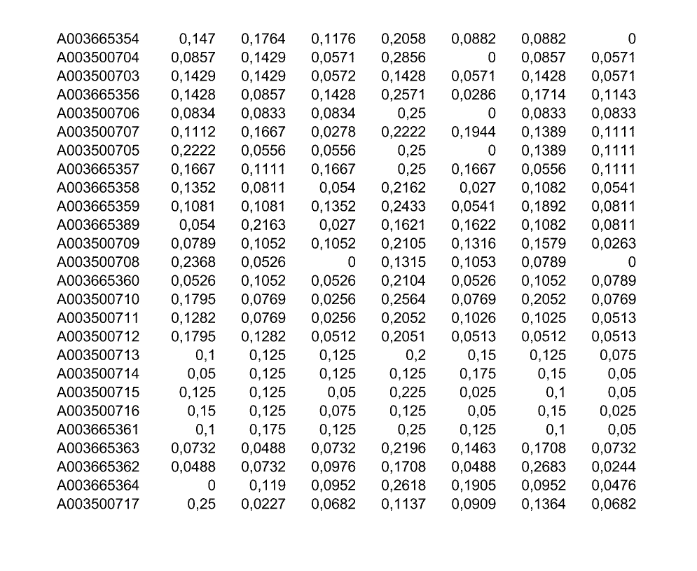

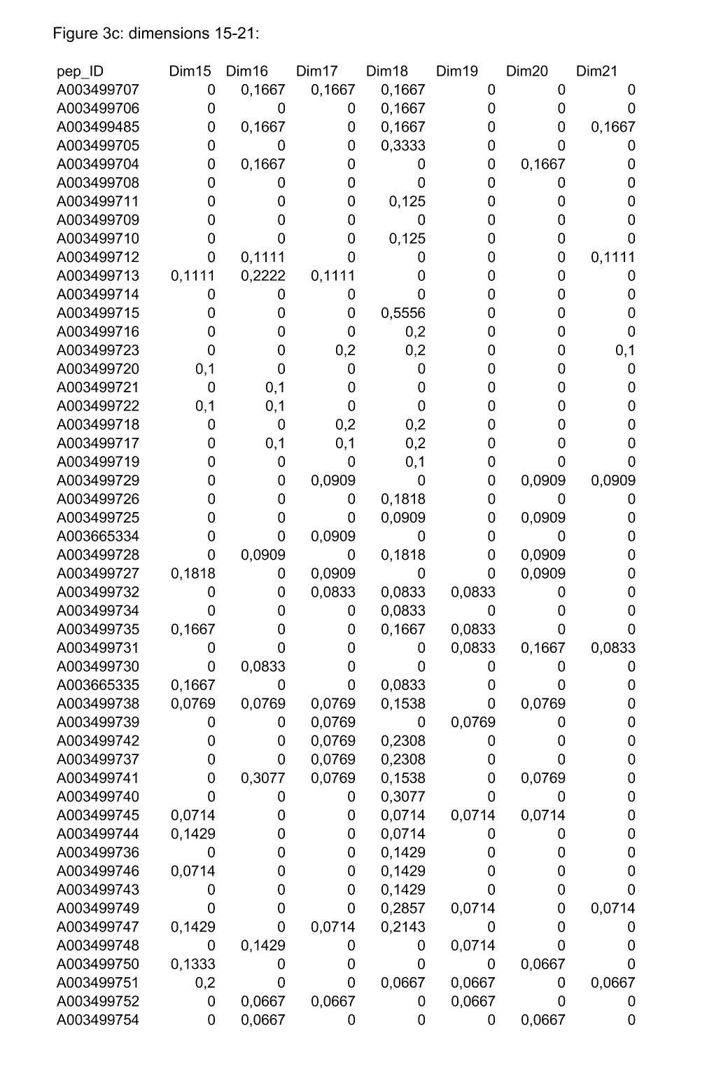

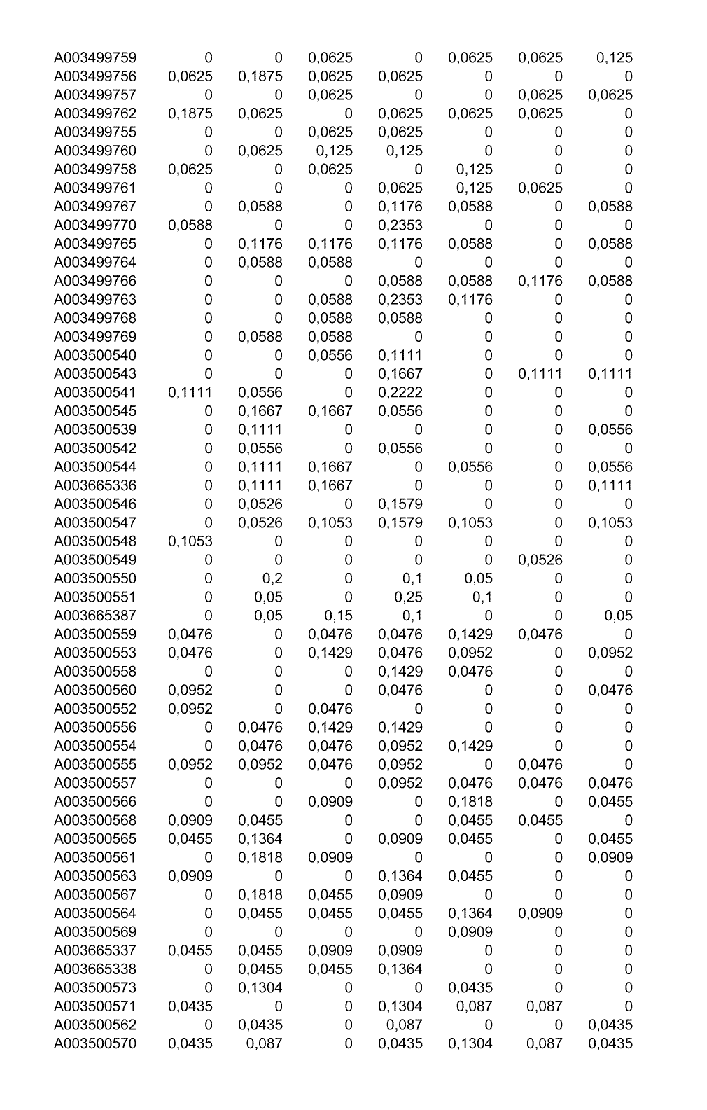

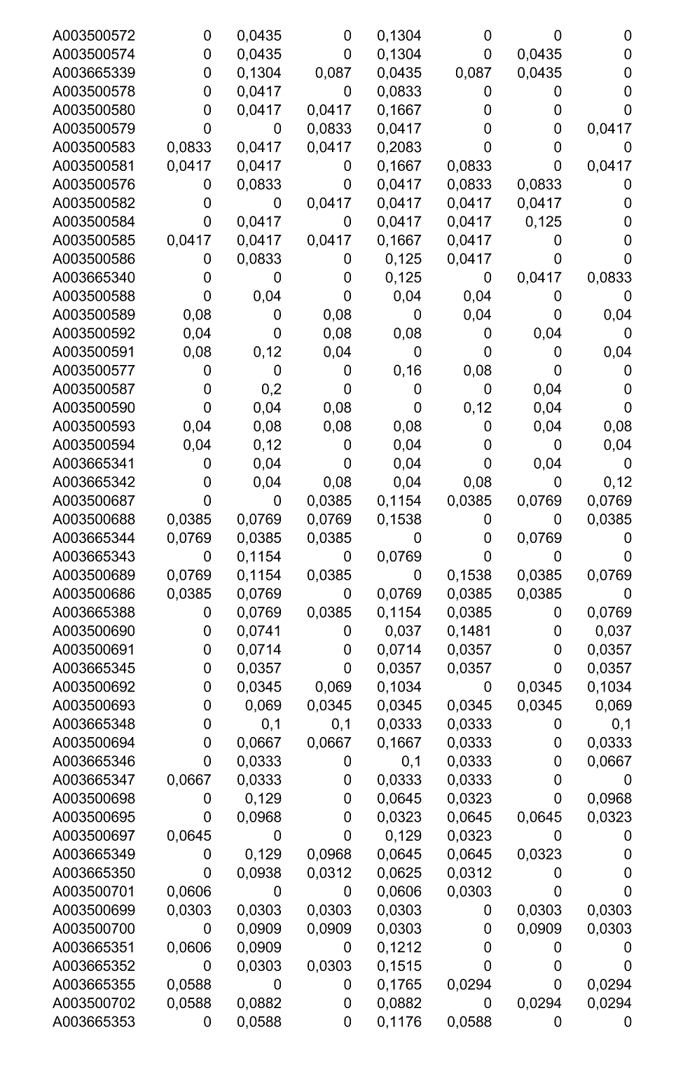

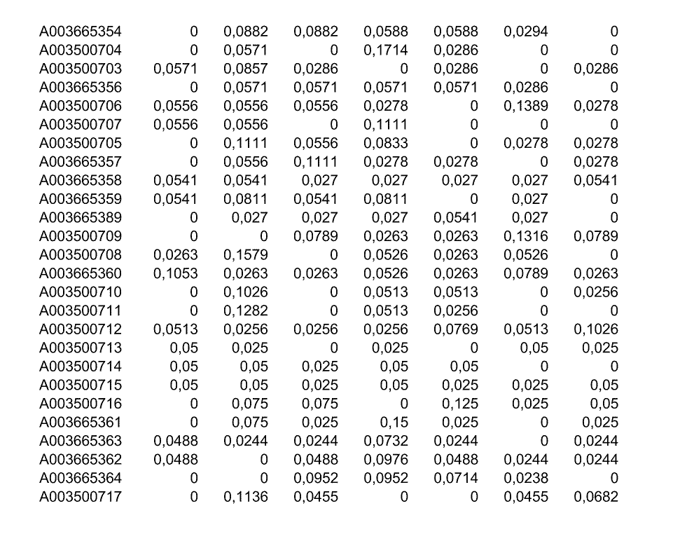

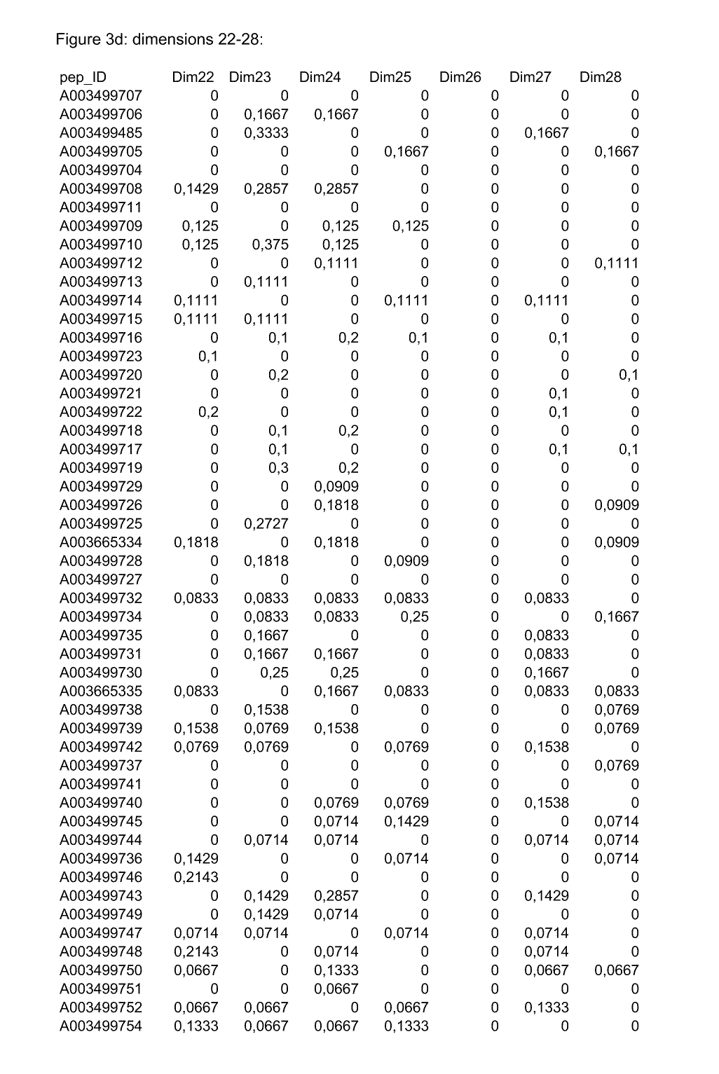

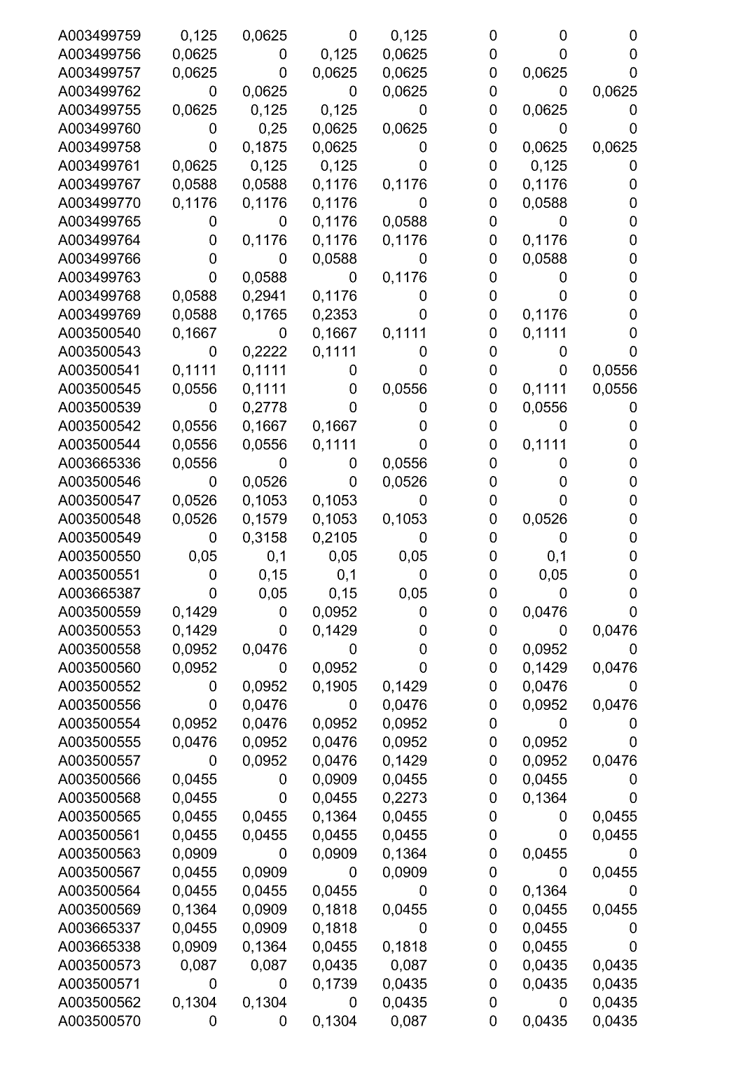

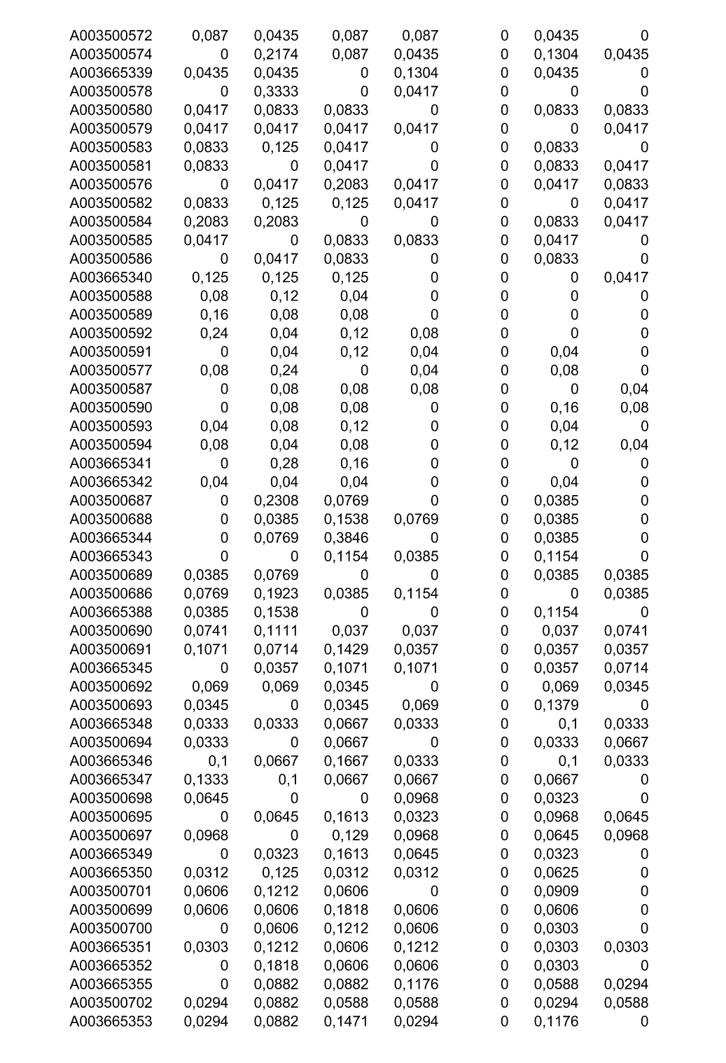

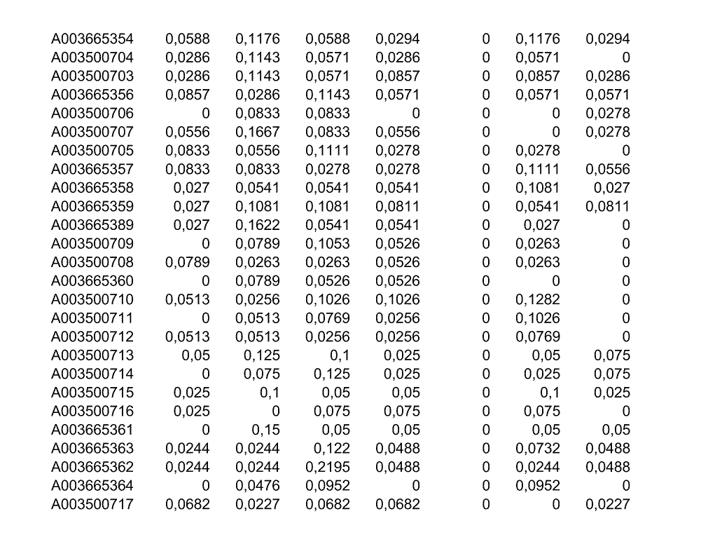

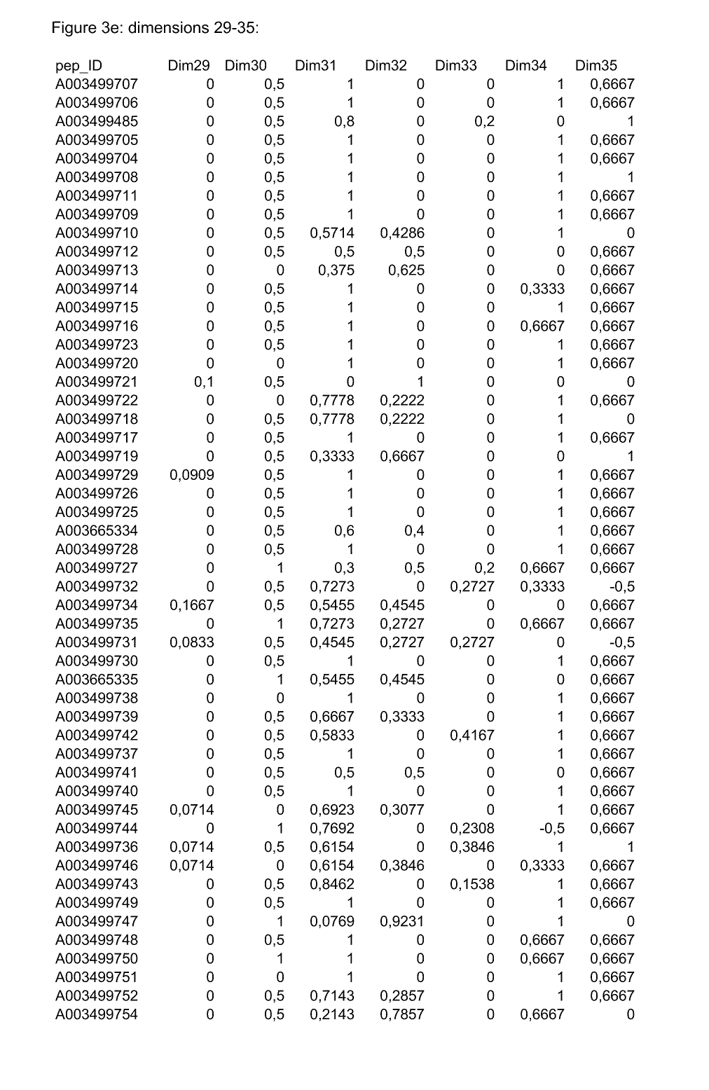

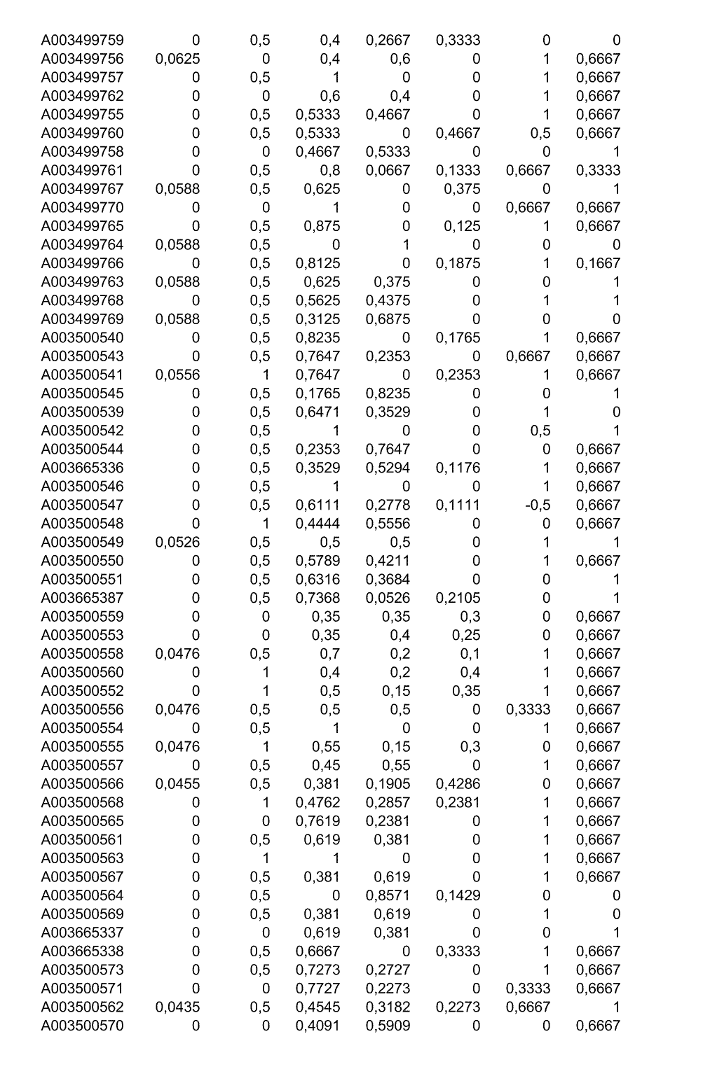

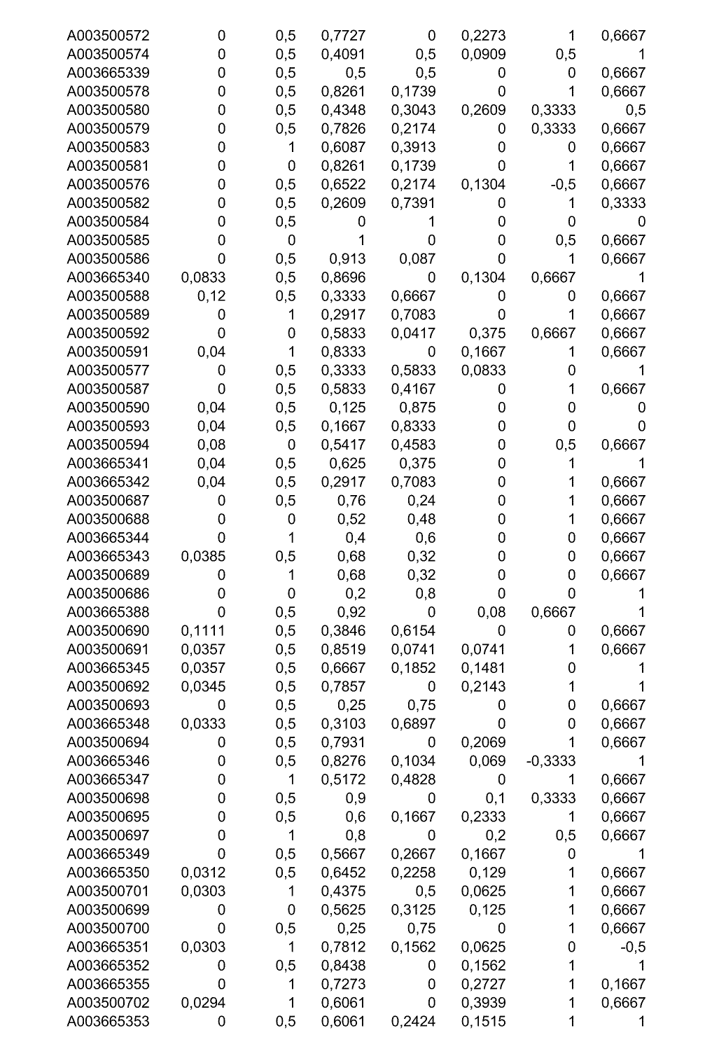

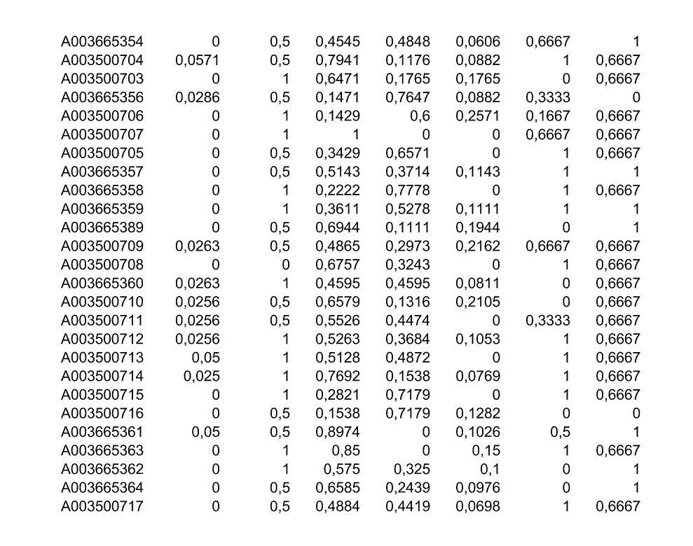

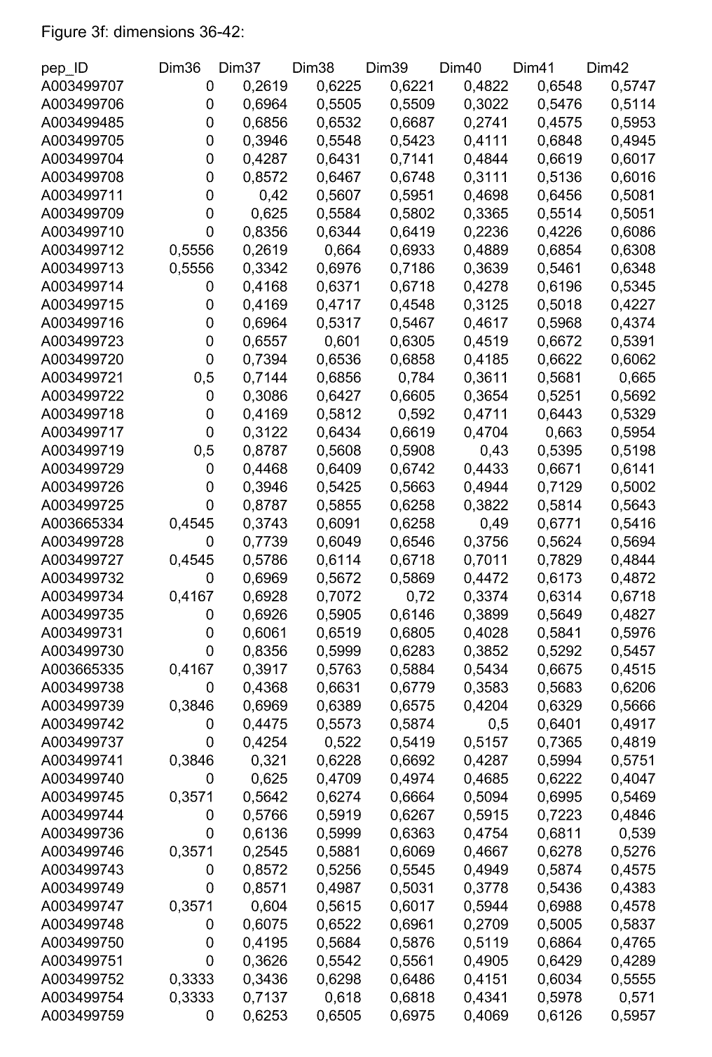

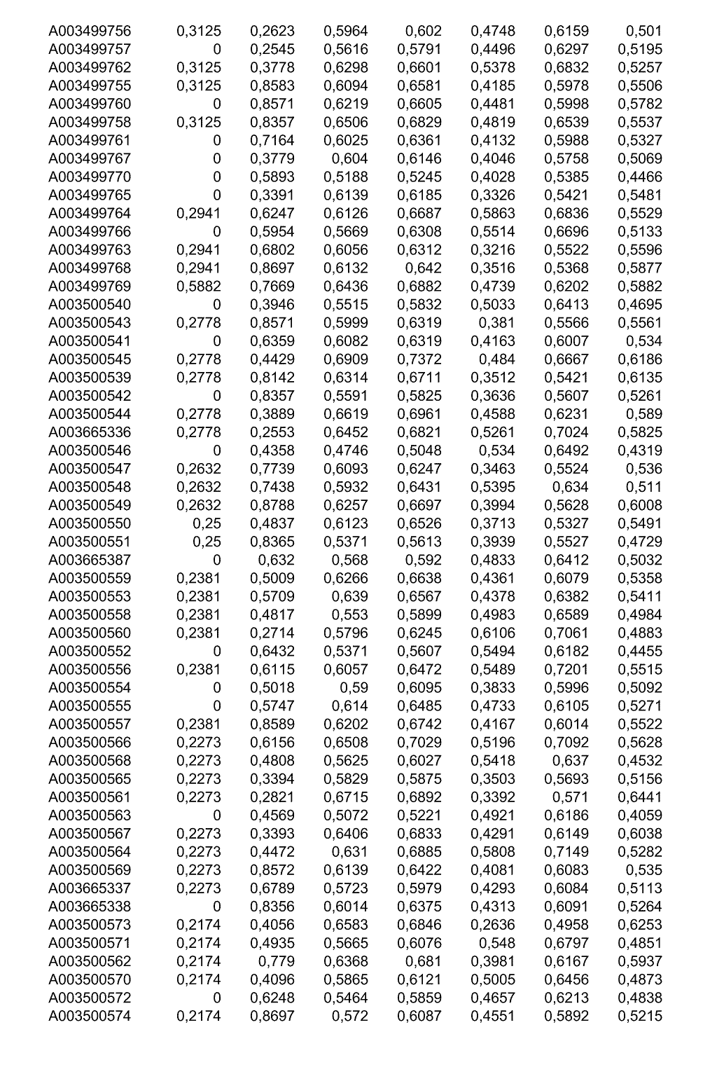

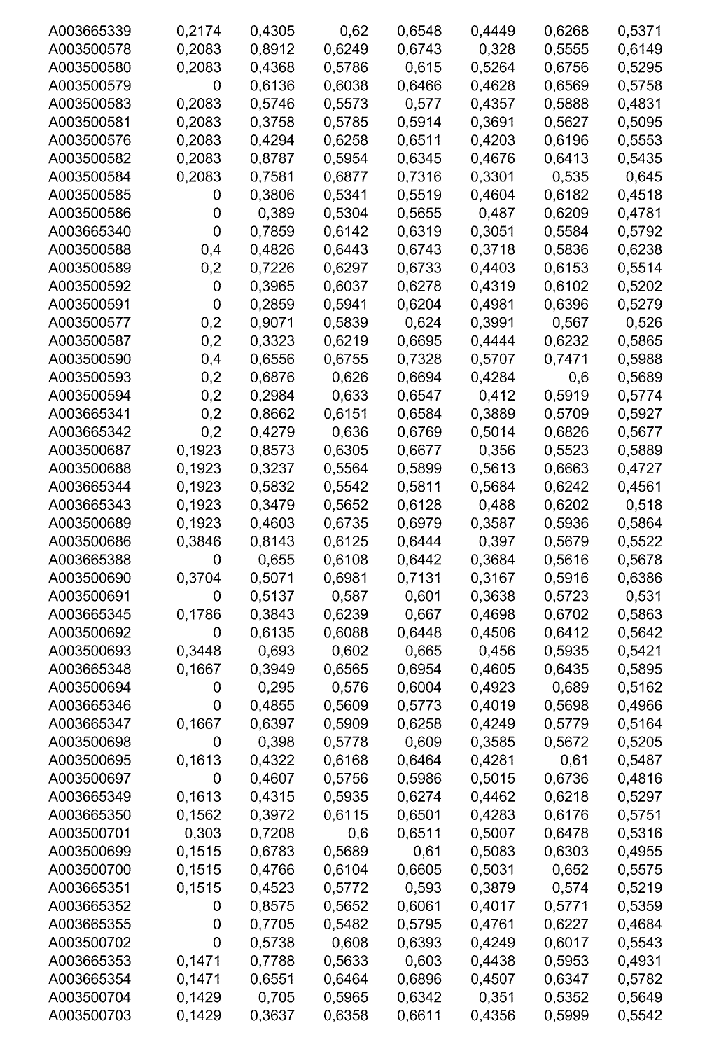

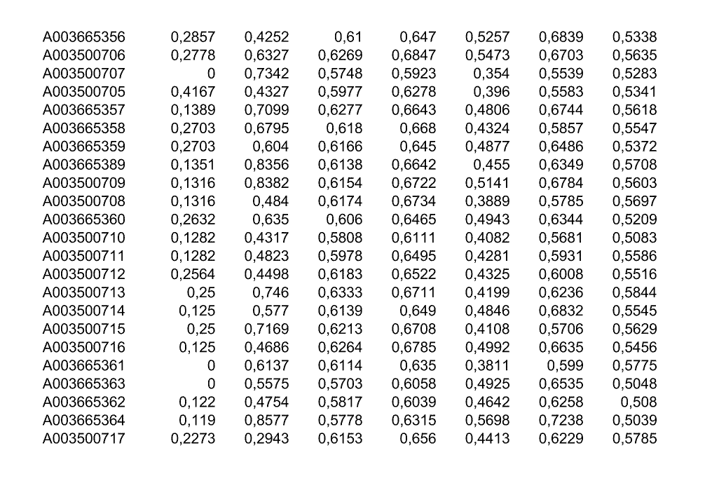

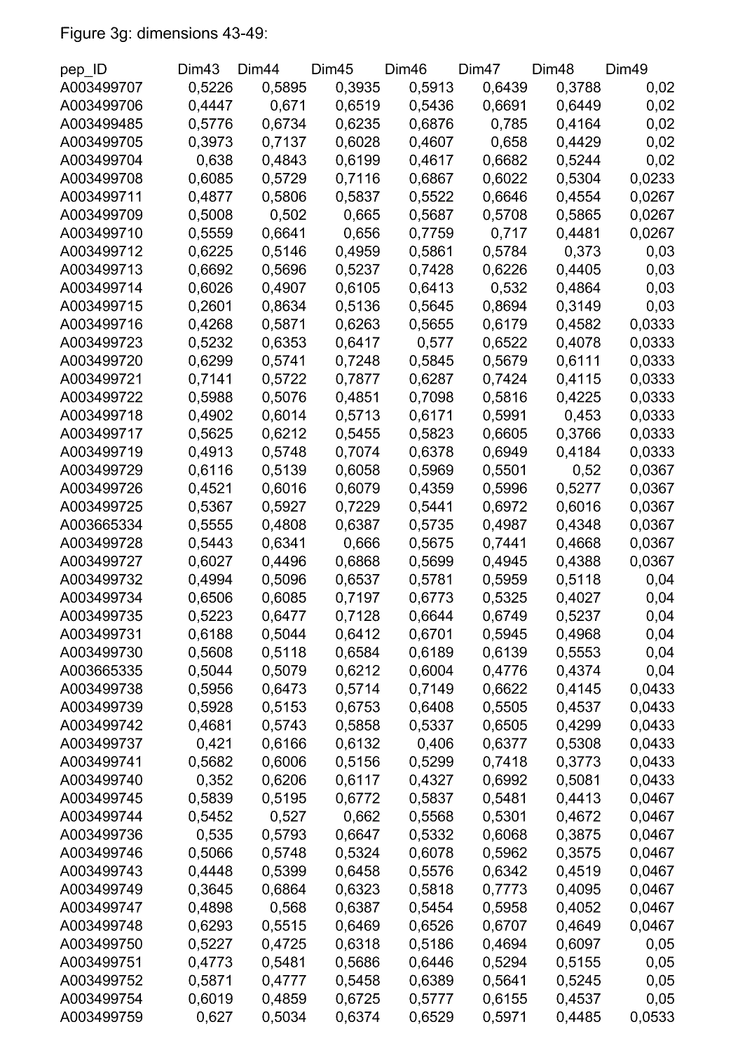

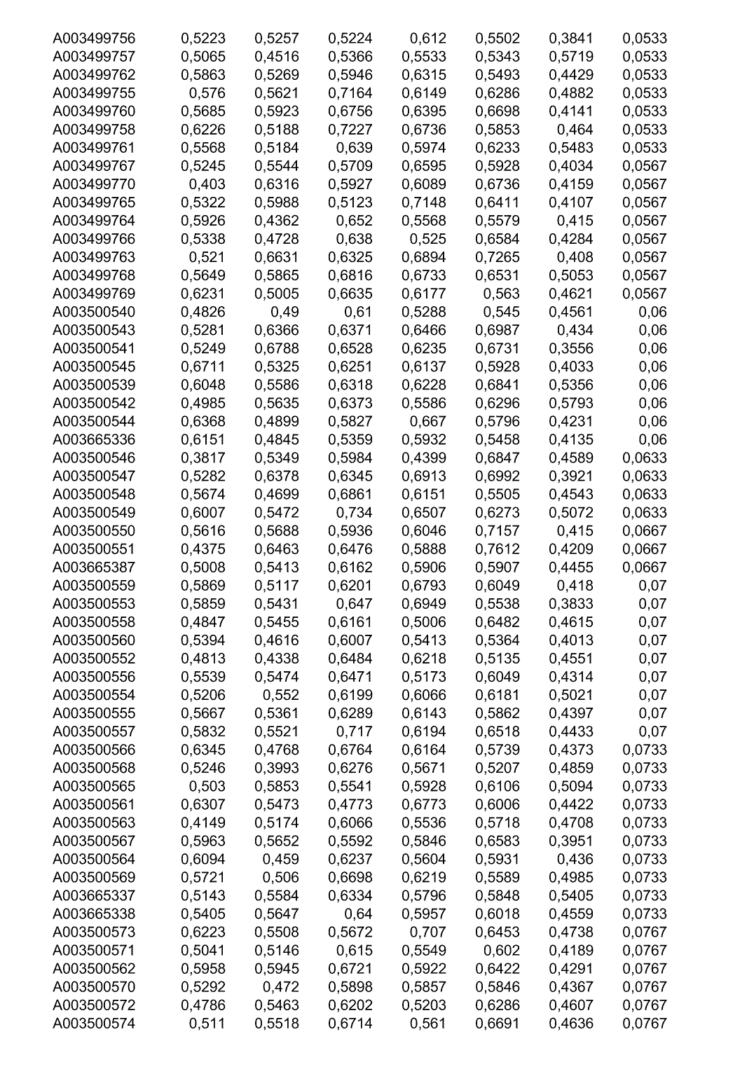

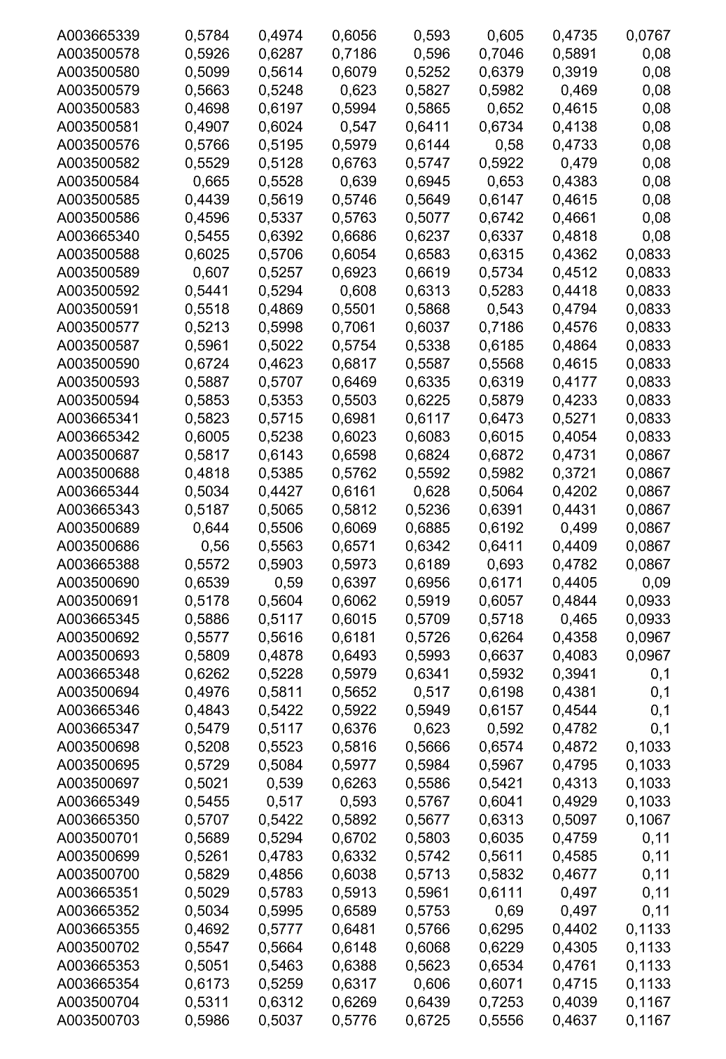

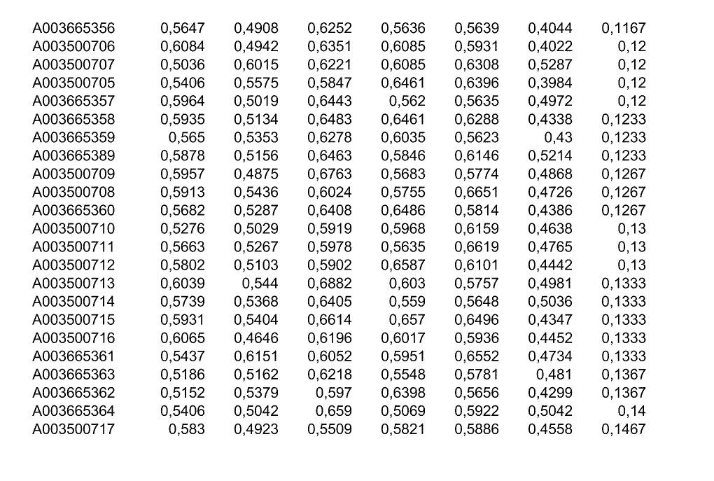

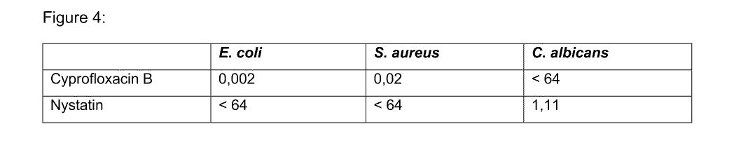

1. A method for identifying bioactive peptides using a binary support vector machine (SVM) based algorithm in a computer based system, the method comprising the steps of:
a) training an SVM algorithm to learn to distinguish between bioactive and non bioactive peptides, said training comprising the steps of:
(i) generating vectors with 49 dimensions, each dimension resulting from the calculation of a molecular descriptor value, for a set of labelled known bioactive and labelled known non bioactive peptides, where the labels indicate whether the peptide is, respectively, bioactive or non bioactive;
(ii) transferring the vector data generated in step (i) to the SVM based algorithm, said algorithm calculating the optimal hyperplane that separates the vectors corresponding to the bioactive peptides and the non bioactive peptides, respectively;
b) providing protein sequences from a publicly available human protein database;
c) predicting secondary structure and cleavage sites within a protein sequence provided in step b) using computational methods; a set of 7 molecular descriptors is calculated based on said prediction step resulting in the generation of peptide fragments;
d) calculating a set of 42 molecular descriptors corresponding to the physico-chemical properties of the peptide fragments generated in step c);
e) transforming the calculated values from step c) into scaled values between 0 and 1 to generate dimensions 1 to 7 of a 49-dimension-vector for each peptide fragment and transforming the calculated values from step d) into scaled values between 0 and 1 to generate dimensions 8 to 49 of said vector for each peptide fragment;
f) presenting the vectors generated in the step e) to the trained SVM algorithm from step a) to measure the distance of each vector to the hyperplane calculated in step a)(ii); and
g) classifying each peptide fragment as bioactive peptide or non bioactive peptide, according to the distance measured in step f).

2. The method of claim 1, wherein dimensions 1 to 7 generated in step e) are: Dimension 1: N-terminal ProP score; Dimension 2: N-terminal Hmcut score; Dimension 3: N-terminal fragment; Dimension 4: C-terminal ProP score; Dimension 5: C-terminal Hmcut score; Dimension 6: C-terminal Hamid score; Dimension 7: C-terminal fragment; and dimensions 8 to 49 generated in step e) are the following: Dimension 8: Percentage of acidic amino acids (E, N, Q) per polypeptide; Dimension 9: Percentage of positively charged amino acids (R, H) per polypeptide; Dimension 10: Percentage of aromatic amino acids (F, Y, W) per polypeptide; Dimension 11: Percentage of aliphatic amino acids (G, V, A, I) per polypeptide; Dimension 12: Percentage of Proline per polypeptide; Dimension 13: Percentage of reactive amino acids (S, T) per polypeptide; Dimension 14: Percentage of Alanine per polypeptide; Dimension 15: Percentage of Cysteine per polypeptide; Dimension 16: Percentage of Glutamic acid per polypeptide; Dimension 17: Percentage of Phenylalanine per polypeptide; Dimension 18: Percentage of Glycine per polypeptide; Dimension 19: Percentage of Histidine per polypeptide; Dimension 20: Percentage of Isoleucine per polypeptide; Dimension 21: Percentage of Asparagine per polypeptide; Dimension 22: Percentage of Glutamine per polypeptide; Dimension 23: Percentage of Arginine per polypeptide; Dimension 24: Percentage of Serine per polypeptide; Dimension 25: Percentage of Threonine per polypeptide; Dimension 26: Percentage of non-canonical amino acid per polypeptide; Dimension 27: Percentage of Valine per polypeptide; Dimension 28: Percentage of Tryptophane per polypeptide; Dimension 29: Percentage of Tyrosine per polypeptide; Dimension 30: Cysteine content; Dimension 31: Percentage of coiled secondary structure per polypeptide; Dimension 32: Percentage of helical secondary structure per polypeptide; Dimension 33: Percentage of random secondary structure per polypeptide; Dimension 34: Score for structure around N-terminal cleavage site; Dimension 35: Score for structure around C-terminal cleavage site; Dimension 36: Number of helical blocks per polypeptide; Dimension 37: Isoelectric point of polypeptide; Dimension 38: Average molecular weight of polypeptide; Dimension 39: Sum of Van-der-Waals forces of each amino acid within polypeptide; Dimension 40: Sum of hydrophobicity values of each amino acid within polypeptide; Dimension 41-48: Mean values calculated based on principle component score vectors of hydrophobic, steric, and electronic properties per polypeptide; Dimension 49: Length of polypeptide.

3. The method of claim 1, wherein protein sequences from step b) are only naturally occurring protein sequences found in the human secretome.

4. The method of claim 1, wherein said bioactive peptides are bioactive peptide hormones derived from precursor hormones.

5. A bioactive peptide selected from the human secretome using the method of claim 1.

6. The bioactive peptide of claim 5, wherein said bioactive peptide is a bioactive peptide hormone.

7. The bioactive peptide of claim 6, wherein said bioactive peptide hormone derives from a precursor protein.

8. The A bioactive peptide of claim 5, having an amino acid sequence selected from the group consisting of: SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38. 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138. 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, and 185.

9. A peptide library comprising bioactive peptides identified using the method of claim 1.

10. The peptide library according of claim 9, wherein said peptide library comprises a bioactive peptide having an amino acid sequence selected from the group consisting of: SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38. 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138. 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, and 185.

11. The peptide library of claim 9, wherein said bioactive peptide is a bioactive hormone.

12. The peptide library of claim 11, wherein said bioactive peptide hormone derives from a precursor protein.

13. A computational device configured to identify bioactive peptides by using a binary support vector machine (SVM) based method, said method comprising the steps of:
a) training an SVM algorithm to learn to distinguish between bioactive and non bioactive peptides, said training comprising the steps of:
(i) generating vectors with 49 dimensions, each dimension resulting from the calculation of a molecular descriptor value, for a set of labelled known bioactive and labelled known non bioactive peptides, where the labels indicate whether the peptide is, respectively, bioactive or non bioactive;
(ii) transferring the vector data generated in step (i) to the SVM based algorithm, said algorithm calculating the optimal hyperplane that separates the vectors corresponding to the bioactive peptides and the non bioactive peptides, respectively;
b) providing protein sequences from a publicly available human protein database;
c) predicting secondary structure and cleavage sites within a protein sequence provided in step b) using computational methods; a set of 7 molecular descriptors is calculated based on said prediction step resulting in the generation of peptide fragments;
d) calculating a set of 42 molecular descriptors corresponding to the physico-chemical properties of the peptide fragments generated in step c);
e) transforming the calculated values from step c) into scaled values between 0 and 1 to generate dimensions 1 to 7 of a 49-dimension-vector for each peptide fragment and transforming the calculated values from step d) into scaled values between 0 and 1 to generate dimensions 8 to 49 of said vector for each peptide fragment;
f) presenting the vectors generated in the step e) to the trained SVM algorithm from step a) to measure the distance of each vector to the hyperplane calculated in step a)(ii); and
g) classifying each peptide fragment as bioactive peptide or non bioactive peptide, according to the distance measured in step f).

14. (canceled)

15. (canceled)

16. A pharmaceutical composition comprising a bioactive peptide as a bioactive agent, wherein the bioactive peptide has an amino acid a sequence selected from the group consisting of: SEQ ID NOs:1-184, and SEQ ID NO:185.


SEQUENCE LISTING (185 sequences)

SEQ ID NO: 1 - protein, 6 aa, Homo sapiens
MLEDGF

SEQ ID NO: 2 - protein, 6 aa, Homo sapiens
GRSPPG

SEQ ID NO: 3 - protein, 6 aa, Homo sapiens
VRVGEN

SEQ ID NO: 4 - protein, 6 aa, Homo sapiens
TGPGTW

SEQ ID NO: 5 - protein, 6 aa, Homo sapiens
GIELKP

SEQ ID NO: 6 - protein, 7 aa, Homo sapiens
QPLRSQR

SEQ ID NO: 7 - protein, 8 aa, Homo sapiens
GALGPDMK

SEQ ID NO: 8 - protein, 8 aa, Homo sapiens
RQPPKATS

SEQ ID NO: 9 - protein, 8 aa, Homo sapiens
HRDRGQAS

SEQ ID NO: 10 - protein, 9 aa, Homo sapiens
FLEDSLLNW

SEQ ID NO: 11 - protein, 9 aa, Homo sapiens
MECLEKRDF

SEQ ID NO: 12 - protein, 9 aa, Homo sapiens
RQMPTMDVK

SEQ ID NO: 13 - protein, 9 aa, Homo sapiens
RRDGQGGGG

SEQ ID NO: 14 - protein, 10 aa, Homo sapiens
SGGSPVRTSM

SEQ ID NO: 15 - protein, 10 aa, Homo sapiens
APFQGNFGLK

SEQ ID NO: 16 - protein, 10 aa, Homo sapiens
ACPPRAPLWR

SEQ ID NO: 17 - protein, 10 aa, Homo sapiens
TLKVYEKKKL

SEQ ID NO: 18 - protein, 10 aa, Homo sapiens
RQDVCDLQKE

SEQ ID NO: 19 - protein, 10 aa, Homo sapiens
SFSGFGSPLD

SEQ ID NO: 20 - protein, 10 aa, Homo sapiens
KERLDFGGWV

SEQ ID NO: 21 - protein, 10 aa, Homo sapiens
VRLSRSAAAR

SEQ ID NO: 22 - protein, 11 aa, Homo sapiens
SPYISDPKNLF

SEQ ID NO: 23 - protein, 11 aa, Homo sapiens
AWPLPGLSPSG

SEQ ID NO: 24 - protein, 11 aa, Homo sapiens
APARPGAPRRI

SEQ ID NO: 25 - protein, 11 aa, Homo sapiens
HSQPAQSFLWA

SEQ ID NO: 26 - protein, 11 aa, Homo sapiens
DLGTKGPRREI

SEQ ID NO: 27 - protein, 11 aa, Homo sapiens
AAPCFICMKLL

SEQ ID NO: 28 - protein, 12 aa, Homo sapiens
TQGHPASAPVFT

SEQ ID NO: 29 - protein, 12 aa, Homo sapiens
HYTTWYTKGSRW

SEQ ID NO: 30 - protein, 12 aa, Homo sapiens
MRPHCKRGPGVC

SEQ ID NO: 31 - protein, 12 aa, Homo sapiens
WSDIHNPVSYRI

SEQ ID NO: 32 - protein, 12 aa, Homo sapiens
NRVSEPRAPSSR

SEQ ID NO: 33 - protein, 12 aa, Homo sapiens
DVCTQCLWPSSG

SEQ ID NO: 34 - protein, 13 aa, Homo sapiens
YGDWERKGRCIDF

SEQ ID NO: 35 - protein, 13 aa, Homo sapiens
PRHSQPAQSFLWA

SEQ ID NO: 36 - protein, 13 aa, Homo sapiens
GPVGVQTFRLDGG

SEQ ID NO: 37 - protein, 13 aa, Homo sapiens
FPGAPPPWLAGGF

SEQ ID NO: 38 - protein, 13 aa, Homo sapiens
FPGAPPPWLAGGF

SEQ ID NO: 39 - protein, 13 aa, Homo sapiens
KKPPTPGGSVGVG

SEQ ID NO: 40 - protein, 14 aa, Homo sapiens
TTCLPKSLYHWGIT

SEQ ID NO: 41 - protein, 14 aa, Homo sapiens
VLLCCPGWRAMVPS

SEQ ID NO: 42 - protein, 14 aa, Homo sapiens
NWTPQAMLYLKGAQ

SEQ ID NO: 43 - protein, 14 aa, Homo sapiens
AAGQDGLLDLCYQQ

SEQ ID NO: 44 - protein, 14 aa, Homo sapiens
GPGASGLRVSSVRS

SEQ ID NO: 45 - protein, 14 aa, Homo sapiens
EAAPGNHGRSAGRG

SEQ ID NO: 46 - protein, 14 aa, Homo sapiens
SPGLCLGFVTQGLC

SEQ ID NO: 47 - protein, 14 aa, Homo sapiens
RKHKPQVSQQEELK

SEQ ID NO: 48 - protein, 15 aa, Homo sapiens
SPCAPPQPSWPCSVI

SEQ ID NO: 49 - protein, 15 aa, Homo sapiens
YCNPAPLCSDGCHPM

SEQ ID NO: 50 - protein, 15 aa, Homo sapiens
DPQHFADMPRVTDVE

SEQ ID NO: 51 - protein, 15 aa, Homo sapiens
ETSPILTEKQAKQLL

SEQ ID NO: 52 - protein, 16 aa, Homo sapiens
SQLQTLIFNPTKNDHL

SEQ ID NO: 53 - protein, 16 aa, Homo sapiens
KSGCTLAEAESFMEQY

SEQ ID NO: 54 - protein, 16 aa, Homo sapiens [sequence omitted]

SEQ ID NO: 55 - protein, 16 aa, Homo sapiens
STEDLPCHLLRICGWC

SEQ ID NO: 56 - protein, 16 aa, Homo sapiens
GGQLPPKVSRRMAFSK

SEQ ID NO: 57 - protein, 16 aa, Homo sapiens
GRLFLRAERRAGGFTS

SEQ ID NO: 58 - protein, 16 aa, Homo sapiens
SMCHRWSRAVLFPAAH

SEQ ID NO: 59 - protein, 16 aa, Homo sapiens
LPSLPVSVQHHPRIGR

SEQ ID NO: 60 - protein, 17 aa, Homo sapiens
TTYVMDVSTNQGSGMEH

SEQ ID NO: 61 - protein, 17 aa, Homo sapiens
AGSGDRCVPAAQRSGQG

SEQ ID NO: 62 - protein, 17 aa, Homo sapiens
STGFMEFDDNEGKHSSK

SEQ ID NO: 63 - protein, 17 aa, Homo sapiens
RLSEALLYLVRAVSTFT

SEQ ID NO: 64 - protein, 17 aa, Homo sapiens
TSDAVHANKLGLPLKII

SEQ ID NO: 65 - protein, 17 aa, Homo sapiens
LTHFLKTHGYDDGGKRG

SEQ ID NO: 66 - protein, 17 aa, Homo sapiens
GRDPSASRARFPQRLGR

SEQ ID NO: 67 - protein, 17 aa, Homo sapiens
LSPLLVSRSEYLFVRSQ

SEQ ID NO: 68 - protein, 18 aa, Homo sapiens
GQSPVTQGVPGTSQLFLS

SEQ ID NO: 69 - protein, 18 aa, Homo sapiens
FGIPMDRIGRNRLSNSRG

SEQ ID NO: 70 - protein, 18 aa, Homo sapiens
TKGCQGRWECQRGALGYL

SEQ ID NO: 71 - protein, 18 aa, Homo sapiens
DVFFEFWVMLRTELEKGQ

SEQ ID NO: 72 - protein, 18 aa, Homo sapiens
RPLLDRRAAREEPVPNRR

SEQ ID NO: 73 - protein, 18 aa, Homo sapiens
HPEPASPRSAGSPARLQR

SEQ ID NO: 74 - protein, 18 aa, Homo sapiens
KVERLFVEKFHQSFSLDN

SEQ ID NO: 75 - protein, 18 aa, Homo sapiens
RNPTDMFEFFANEQLLLL

SEQ ID NO: 76 - protein, 19 aa, Homo sapiens
REPRAALAAPATLGPGAAG

SEQ ID NO: 77 - protein, 19 aa, Homo sapiens
FRFQEFLAHGGSNNSRKGH

SEQ ID NO: 78 - protein, 19 aa, Homo sapiens
KRCARLLTRLAVSPLCSQT

SEQ ID NO: 79 - protein, 19 aa, Homo sapiens
ARARPASSLASSYIRRPRL

SEQ ID NO: 80 - protein, 20 aa, Homo sapiens
GGSKEALHGTERKEQLVREV

SEQ ID NO: 81 - protein, 20 aa, Homo sapiens
ASKEGGHGRGVLSHAARAGR

SEQ ID NO: 82 - protein, 20 aa, Homo sapiens
AEAGLPSSRSFMGFAAPFTN

SEQ ID NO: 83 - protein, 21 aa, Homo sapiens
DISVLFLQSDCQHHHDLKKQG

SEQ ID NO: 84 - protein, 21 aa, Homo sapiens
ISAFFQHFQNSGSLLWCQNHK

SEQ ID NO: 85 - protein, 21 aa, Homo sapiens
THGPLQAAVALPDPLRGYVGQ

SEQ ID NO: 86 - protein, 21 aa, Homo sapiens
GVALSCWQLDPSVLVLCGQNL

SEQ ID NO: 87 - protein, 21 aa, Homo sapiens
ASVTTCTCAFRAARASPALSS

SEQ ID NO: 88 - protein, 21 aa, Homo sapiens
AVLGFTFVEGFAKPLPWYARG

SEQ ID NO: 89 - protein, 21 aa, Homo sapiens
GQHSTESHRHLPAFPPQTLGG

SEQ ID NO: 90 - protein, 21 aa, Homo sapiens
QCLVFVTPQRRGTCEKYSGEI

SEQ ID NO: 91 - protein, 21 aa, Homo sapiens
RRTSTVVHKGTKRMLKNPGWI

SEQ ID NO: 92 - protein, 22 aa, Homo sapiens
FLLFLPYHTVHFSLSLKNHHQL

SEQ ID NO: 93 - protein, 22 aa, Homo sapiens
SQTTHTAIVTPACLVTCVSEKP

SEQ ID NO: 94 - protein, 22 aa, Homo sapiens
GHSGASPTPGPQELERENSWCP

SEQ ID NO: 95 - protein, 22 aa, Homo sapiens
SPEDEEKNFDQTRFLEDSLLNW

SEQ ID NO: 96 - protein, 22 aa, Homo sapiens
DTGASTTGHPVLSQPACAPCGQ

SEQ ID NO: 97 - protein, 22 aa, Homo sapiens
SLGLRTFRKDLWEEAELGQTLE

SEQ ID NO: 98 - protein, 22 aa, Homo sapiens
RIHVFLLDLILPVHGMMQSVHE

SEQ ID NO: 99 - protein, 22 aa, Homo sapiens
WPRVPQPQHSAQSLAWSHLMTR

SEQ ID NO: 100 - protein, 22 aa, Homo sapiens
QFVSCAEQFRSPGSLSPGPKPP

SEQ ID NO: 101 - protein, 22 aa, Homo sapiens
QGLTQTPTEMQRVSLRFGGPMT

SEQ ID NO: 102 - protein, 23 aa, Homo sapiens
DEDTPRKDHVKTWREDSQLQAKE

SEQ ID NO: 103 - protein, 23 aa, Homo sapiens
SLGSTSSGDLGHILCPLVSWHKI

SEQ ID NO: 104 - protein, 23 aa, Homo sapiens
APQRLLERRNWTPQAMLYLKGAQ

SEQ ID NO: 105 - protein, 23 aa, Homo sapiens
RHAHNIGETWISALSEPVCHTLS

SEQ ID NO: 106 - protein, 23 aa, Homo sapiens
KQPLGPPGLQLAGSMESTRTVAK

SEQ ID NO: 107 - protein, 23 aa, Homo sapiens
TVITSPREVVRAARRWAAGSPGG

SEQ ID NO: 108 - protein, 23 aa, Homo sapiens
AATKFGPETAIPRELMFHEVHQT

SEQ ID NO: 109 - protein, 24 aa, Homo sapiens
VGARGRRRPRALLRPLPETPPRRP

SEQ ID NO: 110 - protein, 24 aa, Homo sapiens
VAALGVLGAEWGPDLSGFWVQLRS

SEQ ID NO: 111 - protein, 24 aa, Homo sapiens
KLPFLNWDAFPKLKGLRSATPDAQ

SEQ ID NO: 112 - protein, 24 aa, Homo sapiens
AVQVAEPLGSCGFQGGPCPGRRRD

SEQ ID NO: 113 - protein, 24 aa, Homo sapiens
SNGHVGSCLQPQGDWKDEVKDAGH

SEQ ID NO: 114 - protein, 24 aa, Homo sapiens
RWIPGSSWPMDVSHHSILETEKRS

SEQ ID NO: 115 - protein, 24 aa, Homo sapiens
QPLQPAFGRLTAASSAILQWHRSP

SEQ ID NO: 116 - protein, 24 aa, Homo sapiens
VHVKQQWDQQRLRDGVIRDIERQI

SEQ ID NO: 117 - protein, 24 aa, Homo sapiens
QPVPTQETGPKAMGDLSCGFAGHS

SEQ ID NO: 118 - protein, 24 aa, Homo sapiens
RKPASAGPVPDLVLGAEEAHGSRL

SEQ ID NO: 119 - protein, 24 aa, Homo sapiens
FPQSGRQWNGYINAYPKSSQPPRG

SEQ ID NO: 120 - protein, 25 aa, Homo sapiens
PLLSRAQQRKRDGPDLAEYYYDAHL

SEQ ID NO: 121 - protein, 25 aa, Homo sapiens
KPNKHSRACQQFLKQCQLRSFALPL

SEQ ID NO: 122 - protein, 25 aa, Homo sapiens
HSGQGTSPQQDTQSFFLCPIMLQRQ

SEQ ID NO: 123 - protein, 25 aa, Homo sapiens
TNSEPSYELPALETAFPVDCRLLCS

SEQ ID NO: 124 - protein, 25 aa, Homo sapiens
AQTGHQVRRVLPAARRGRLHARAPG

SEQ ID NO: 125 - protein, 25 aa, Homo sapiens
RELSPTAPKWLEEAEERLTLRSIPL

SEQ ID NO: 126 - protein, 25 aa, Homo sapiens
LPELLRIYFLVLPVFSHWHVSHVWL

SEQ ID NO: 127 - protein, 25 aa, Homo sapiens
FNSGSVFRNELLPLFKEYGKSKIQR

SEQ ID NO: 128 - protein, 25 aa, Homo sapiens
KRGKQVCADPSESWVQEYVYDLELN

SEQ ID NO: 129 - protein, 25 aa, Homo sapiens
ARARPASSLASSYIRRPRLREPPGL

SEQ ID NO: 130 - protein, 25 aa, Homo sapiens
RHNLQLLNDMAGRLYHFSEVLPNLF

SEQ ID NO: 131 - protein, 26 aa, Homo sapiens
VVDHPKRRFGIPMDRIGRNRLSNSRG

SEQ ID NO: 132 - protein, 26 aa, Homo sapiens
GETGEGLSLAFLSSLMFTSRNGLVGC

SEQ ID NO: 133 - protein, 26 aa, Homo sapiens
SERCFSILALSVCSASISSSSSSMRA

SEQ ID NO: 134 - protein, 26 aa, Homo sapiens
DVLEAEKSKVKTPVDYASGESLLPGL

SEQ ID NO: 135 - protein, 26 aa, Homo sapiens
AEEQFLEHWLNPHCKPHCDRNRIHPV

SEQ ID NO: 136 - protein, 26 aa, Homo sapiens
AEAARALQRRCSQATGPIWRTLRTHE

SEQ ID NO: 137 - protein, 26 aa, Homo sapiens
APKLGQLEDNHVRPFPADGRVRVGEN

SEQ ID NO: 138 - protein, 27 aa, Homo sapiens
SREAVEQWRQWHYDGLHPSYLYNRHHT

SEQ ID NO: 139 - protein, 28 aa, Homo sapiens
HPSPSVSAQEGPGRQPTALQNYSHWARE

SEQ ID NO: 140 - protein, 28 aa, Homo sapiens
SLVTSLYLPNTEDLSLWLWPKPDLHSGT

SEQ ID NO: 141 - protein, 29 aa, Homo sapiens
QIPAPQGAVLVQREKDLPNYNWNSFGLRF

SEQ ID NO: 142 - protein, 29 aa, Homo sapiens
DAGVLEKVLKIKEQNVHNKTASTFLKKDV

SEQ ID NO: 143 - protein, 30 aa, Homo sapiens
SVYNGLELNTWMKVERLFVEKFHQSFSLDN

SEQ ID NO: 144 - protein, 30 aa, Homo sapiens
GWGQLLPPNHLFVMEEGGWGSSLAADGFPP

SEQ ID NO: 145 - protein, 30 aa, Homo sapiens
LAVGSNSPSGQSRDGATLQNAHPQVESWVP

SEQ ID NO: 146 - protein, 30 aa, Homo sapiens
QLPALPQKCQDVHQPLARARSRQSTVTGEC

SEQ ID NO: 147 - protein, 31 aa, Homo sapiens
EAGAVKEHELTGQNNQMPPEEPKPLPTKTAN

SEQ ID NO: 148 - protein, 31 aa, Homo sapiens
QPNVVEARWIPGSSWPMDVSHHSILETEKRS

SEQ ID NO: 149 - protein, 31 aa, Homo sapiens
ASSAQVSVWLTPGTTQHLPWMGLPGQGCCWS

SEQ ID NO: 150 - protein, 31 aa, Homo sapiens
QHITLHSESLPSAPGRESFTEVFPSKFPEGL

SEQ ID NO: 151 - protein, 32 aa, Homo sapiens
AADPELPRPFLRHVYPELRPGETSLLVGDAQR

SEQ ID NO: 152 - protein, 33 aa, Homo sapiens
SRVPGPSLYPCPLLQRHLSQGRVRLCALVAKDM

SEQ ID NO: 153 - protein, 33 aa, Homo sapiens
SSTVIPKSEVSTALCSLGLQLNMASPSRARFPQ

SEQ ID NO: 154 - protein, 33 aa, Homo sapiens
DPKPDFPKFLSLLGTEIIENAVEFILRSMSRST

SEQ ID NO: 155 - protein, 33 aa, Homo sapiens
AETGTPTSPRCEPGEATPAVRDGCASWPRQGYL

SEQ ID NO: 156 - protein, 33 aa, Homo sapiens
RPRGRRGARVTDKEPKPLLFLPAAGAGRTPSGS

SEQ ID NO: 157 - protein, 34 aa, Homo sapiens
NTGPNRGCGSKTHTRTRPGAALCSSGPWLGLVVM

SEQ ID NO: 158 - protein, 34 aa, Homo sapiens
QDLTWNKYQAEESITCRGEGSPWRLKVGARCLAP

SEQ ID NO: 159 - protein, 34 aa, Homo sapiens
KVPSLREAMEKVPSQGSVPHGAVRLKGLTGSSPH

SEQ ID NO: 160 - protein, 34 aa, Homo sapiens
LPSLPVSVQHHPRIGRDVFFEFWVMLRTELEKGQ

SEQ ID NO: 161 - protein, 35 aa, Homo sapiens
NEDTYKGAYSGVGLALGKDSHLGRKDKVERGQRRK

SEQ ID NO: 162 - protein, 35 aa, Homo sapiens
TVMWCNLECARTTSEMSLRRDPQHFADMPRVTDVE

SEQ ID NO: 163 - protein, 35 aa, Homo sapiens
QSWEVGYSWLQPALESGFFTMTLAQQHVLALHAIS

SEQ ID NO: 164 - protein, 36 aa, Homo sapiens
ELSIKCINIFDLKIWMALGASRASILRMECLEKRDF

SEQ ID NO: 165 - protein, 36 aa, Homo sapiens
LPGESGSCEDGQSAPAQPPRRRTGTRACPPRAPLWR

SEQ ID NO: 166 - protein, 36 aa, Homo sapiens
VSVGTMQMAGEEASEDAKQKIFMQESDASNFLKRRG

SEQ ID NO: 167 - protein, 36 aa, Homo sapiens
APGRVNALQEPFFQAWFQVAVMTAPPESRHLPWVPF

SEQ ID NO: 168 - protein, 37 aa, Homo sapiens
GCPSRWCRDAGVLEKVLKIKEQNVHNKTASTFLKKDV

SEQ ID NO: 169 - protein, 37 aa, Homo sapiens
RPVCTASAGSLPFAFCMVSTTMRSWWEIRGWEEQRLG

SEQ ID NO: 170 - protein, 37 aa, Homo sapiens
VVLSRHQAPFDPRPLPTPLLLLTPRSARILDRGHAEM

SEQ ID NO: 171 - protein, 38 aa, Homo sapiens
LPNGISSRVMHLPYPFIFIISPPLKKLSIFARTLTNRN

SEQ ID NO: 172 - protein, 38 aa, Homo sapiens
PDLTIPEIPPKCGELKTELLGLKERKHKPQVSQQEELK

SEQ ID NO: 173 - protein, 38 aa, Homo sapiens
RIMGENTSYDDPCKGTICRSHRALMRKAAPCFICMKLL

SEQ ID NO: 174 - protein, 39 aa, Homo sapiens
TVTQEAKEGSHADVSSVPVYPLNQTEKTTGKSERMHVAP

SEQ ID NO: 175 - protein, 39 aa, Homo sapiens
QVPHPRKKELELRDVLEAEKSKVKTPVDYASGESLLPGL

SEQ ID NO: 176 - protein, 39 aa, Homo sapiens
MPANRLSCYRKILKDHNCHNLPEGVADLTQIDVNVQDHF

SEQ ID NO: 177 - protein, 40 aa, Homo sapiens
APARWEWLYSIYRKGTKAQRRNVLRSPCAPPQPSWPCSVI

SEQ ID NO: 178 - protein, 40 aa, Homo sapiens
THPYLPRTLCVASEHCFSWRPLELWLPSRAWPLPGLSPSG

SEQ ID NO: 179 - protein, 40 aa, Homo sapiens
ARRGCPSRWCRDAGVLEKVLKIKEQNVHNKTASTFLKKDV

SEQ ID NO: 180 - protein, 40 aa, Homo sapiens [sequence omitted]

SEQ ID NO: 181 - protein, 40 aa, Homo sapiens
SYTFWREDYEGVGWGRDVRGPGTHEPLSAGRLLAPPSRPN

SEQ ID NO: 182 - protein, 41 aa, Homo sapiens
PSSTSKAPWSLWEPTGQLGFPHCPAVRNGAVKLDCLSPVLP

SEQ ID NO: 183 - protein, 41 aa, Homo sapiens
ISDTSQLTCCSHWDPSSSHDKAVGLSPMWFGLFGGISNLRS

SEQ ID NO: 184 - protein, 42 aa, Homo sapiens
AVLPGMFLAPPLHSPVLPGMFLAPPLHSPVLISHFSKVGFRG

SEQ ID NO: 185 - protein, 44 aa, Homo sapiens
SPNPQLLTIPEAATILLASLQKSPEDEEKNFDQTRFLEDSLLNW